(19) United States
(12) Patent Application Publication
(10) Pub. No.: US 2025/0256044 A1
(43) Pub. Date: Aug. 14, 2025

(54) Nicotine Delivery Systems

(71) Applicant: BREATHEASY CO., D/B/A MIIST THERAPEUTICS, Alameda, CA (US)

(72) Inventors: Eric Ezerins, Alameda, CA (US); Jeffrey Schuster, Alameda, CA (US); Dalton Signor, Alameda, CA (US)

(21) Appl. No.: 18/725,100

(22) PCT Filed: Dec. 29, 2022
(86) PCT No.: PCT/US2022/082580

§ 371 Date: Jun. 27, 2024

Related U.S. Application Data
(60) Provisional application No. 63/332,112, filed on Apr. 18, 2022.
(60) Provisional application No. 63/388,509, filed on Jul. 12, 2022.
(60) Provisional application No. 63/418,442, filed on Oct. 21, 2022.

(51) Int. Cl. A61M 15/00; A61M 16/00

(52) U.S. Cl. CPC A61M 15/001; A61M 15/0066; A61M 15/0085; A61M 2016/0024; A61M 2016/0039

(57) ABSTRACT

Nicotine delivery devices and systems, particularly smoking cessation systems are provided by the present disclosure. Such devices and systems produce a nicotine-containing aerosol for inhalation that mimics smoking. The devices are capable of being adapted to control the amount of nicotine delivered to taper the amount of nicotine delivered to the user over time. Methods for treating patients to stop smoking are also disclosed. The devices, systems and methods can also be used to deliver other drugs disclosed herein, such as opioids (e.g., fentanyl).

### CROSS-REFERENCE TO RELATED APPLICATIONS

This application claims priority to U.S. Provisional Application Ser. No. 63/294,498 filed on Dec. 29, 2021; 63/332,112 filed on Apr. 18, 2022; 63/388,509 filed on Jul. 12, 2022; and 63/418,442 filed on Oct. 21, 2022, which are incorporated herein by reference in their entireties to the full extent permitted by law.

### TECHNICAL FIELD

The present disclosure relates to methods and devices for inhalation drug delivery. Preferred devices are inhalation systems which create an experience that is similar to smoking and include, but are not limited to, devices with arrays of nozzles for forming a nicotine containing aerosol. Preferably, the inhalation systems control the amount of nicotine delivered by reducing it essentially to zero over a predetermined amount of time.

### BACKGROUND

The present disclosure generally relates to devices, systems, and methods for promoting smoking cessation and the treatment and management of tobacco and e-cigarette addiction. Tobacco addiction via cigarettes directly impacts 34.2 million people in the United States, leading to over 480,000 preventable deaths and costing the US economy over $891 billion annually. Tobacco addiction is a chronic disease and various agents and methods have been attempted to treat the disease. Today, there are seven FDA-approved medication types for the treatment of tobacco addiction: nicotine replacement therapy (NRT) gums, lozenges, patches, a buccal inhaler, and a nasal spray, as well as well as the prescription drugs varenicline and bupropion.

The most common method used to manage a smoker's tobacco addiction is NRT, and within that product class the most commonly used medication is NRT gum. However, this product has a failure rate over 91% at one year from the outset of abstinence, clearly highlighting the need for new a new pharmacotherapeutic treatment that has higher efficacy (see https://health.gov/healthypeople/objectives-and-data/browse-objectives/tobacco-use/increase-successful-quit-attempts-adults-who-smoke-tu-14).

Non-FDA approved products, such as e-cigarettes and vaping devices that use heat to form a vapor which condenses to an aerosol, are available as smoking replacement devices, but have not been approved for use as smoking cessation products. Available data suggest that over 50% of e-cigarette users also smoke cigarettes, increasing their exposure to harmful tobacco combustion products rather than helping them quit their tobacco addiction. E-cigarettes do not have a sufficiently good safety profile to promote their use as a medical treatment for tobacco addiction in the US, as the use of heat in the formation of the aerosol is known to create carcinogenic compounds including formaldehyde and acetaldehyde. Accordingly, there is a need in the art for a safe and effective inhalation for use as a smoking cessation therapeutic that avoids the disadvantages of oral medications and e-cigarettes.

Aqueous inhalation solutions have many desirable properties. Most existing aqueous inhalation systems are single dose, wherein the user pours formulation from a vial into a container, and then breathes through the device for a period of several minutes to get a dose, followed by an extensive cleaning procedure. These products are not useful for application that require immediate availability when needed, for example pain or craving. Only one system, the Respimat inhaler (Boehringer Ingelheim) delivers multiple doses without filling or cleaning. However, Respimat is an all mechanical inhaler, and thus cannot perform such functions as dose control, usage monitoring, and the like. Therefore, there is a need for an easy to use, multidose, aqueous inhalation system that has the ability to control the delivered dose.

### SUMMARY

The present disclosure is directed to smoking cessation therapy and more particularly, a smoking cessation system comprising at least one of (a) a portable smoking cessation device; (b) firmware contained in the device for controlling the device; (c) a formulation container; and (d) a companion application. In one embodiment, the system creates a patient experience that is similar to smoking and controls the amount of nicotine administered reducing it essentially to zero over a predetermined amount of time, for example about 10 to 16 weeks, about 13 weeks, about 20 to 32 weeks, or about 26 weeks, or about 2 months to 4 months, about 3 months, about 5 to 7 months, about 6 months.

The present disclosure provides in certain embodiments a smoking cessation inhaler, comprising: a durable part (or system or element) of the inhaler comprising a controller and a multidose cartridge configured to be separably attached to the durable part of the inhaler wherein the cartridge comprises (a) an aerosolizer; (b) a formulation chamber containing a formulation comprising an active pharmaceutical ingredient; (c) a debubbler; and (d) a filter. In certain aspects, the durable part of the inhaler further comprises a pump motor assembly and the cartridge comprises a pump head assembly.

In one embodiment, the pump motor assembly comprises an element chosen from one or more of: a motor; a gearhead; an eccentric element; a rotor which circularly translates at least one roller; and an alignment cam. The pump head assembly may comprise an element chosen from one or more of: a support surface; a ring; and a section of tubing. The inhaler may further comprise a recirculation system comprising one or more elements selected from: a debubbler; a debubbler chamber; a pocket; an aerosolizer; a filter; and a pump head.

In another embodiment, the cartridge comprises a filter capable of removing bacteria from the formulation. Such filter may have a pore size of about 5 μm or less, or about 2 μm or less, or about 1 μm or less.

The present disclosure provides for in certain aspects an inhaler that contains multiple doses of an aqueous formulation, for example a solution or suspension of one or more active pharmaceutical ingredients.

The present disclosure also provides in certain aspects that the active pharmaceutical ingredient is an addictive substance; and/or a substance to which a chronic user will develop tolerance. In one embodiment, the amount of the active pharmaceutical ingredient (API) delivered is repeatedly adjusted by the controller wherein the adjustment comprises one or more of: (a) an increase in the amount of active pharmaceutical ingredient delivered; (b) a decrease in the amount of active pharmaceutical ingredient delivered; and (c) a period of increase of active pharmaceutical ingredient delivered followed by a period of decrease of active pharmaceutical ingredient delivered.

Although a significant aspect of the present disclosure relates to smoking cessation devices, systems, formulations, and methods, and describes formulations containing nicotine, other active pharmaceutical ingredients can be delivered using the disclosed system. Such non-nicotine APIs may or may not also include nicotine to facilitate smoking cessation or for use in smoking or vaping replacement. It will also be understood that the system can be used in indications other than smoking cessation, and active compounds other than nicotine or other drugs for smoking may be used. While large molecular weight compounds greater than 10 kDa for systemic effect may be delivered, preferred applications include compounds of any molecular weight for topical or targeted lung treatment, and small molecular weight compounds of less than or about 10 kDa, preferably less than or about 5 kDa, more preferably less than or about 1 kDa, for systemic effect, preferably more rapid systemic effect as compared to other routes of delivery including, but not limited to, subcutaneous, transdermal, intradermal, oral, buccal, ocular, or nasal.

In some embodiments, the treatment applications employing the aerosol device disclosed herein are those that require changes to the dose over time or personalization of dose for individual patients. Changes may include tapering to avoid withdrawal symptoms of legal or illegal, medical or recreational, compounds. Changes may include ramping up, for example due to the increased tolerance over time of compounds including, but not limited to, opioids, for example fentanyl or morphine and pharmaceutically acceptable salts thereof. Changes may also include doses selected by a patient (i.e., self-titration) or caregiver, for example titration of pain medication strength based on perceived pain, or titration of diabetes medications including, but not limited to, insulins or glucagon-like peptides, based on measured blood glucose and/or expected prandial intake.

By way of a non-limiting example, fentanyl or salts thereof may be used with the aerosol device disclosed herein. In one embodiment, the fentanyl is inhaled from the device ad libitum, but with a lockout period between doses to prevent overdoses. A starting maximum allowed dose of about 10-40 μg per hour, or about 20-30 μg/hr, or about 25 μg/hr may be ramped up to a maximum of from about 25-100 mcg/hr, or about 35-65 mcg/hr, or about 50 mcg/hr. The dose escalation may occur over a period of time ranging from about 1 week to 10 weeks, or 2 weeks to 5 weeks, or about 3 weeks. When it is desired to end the therapy, the dose may be ramped down to about zero at a rate of about 2% to about 25% per week, or at a rate of about 3.5% to about 20% per week, or at a rate of about 5% to about 15% per week. This ramping down of the dose may be used to avoid addiction and abuse opioids such as fentanyl.

The present disclosure further provides a companion application capable of one or more of: controlling communication with the smoking cessation device, wherein the communication is preferably wireless, more preferably Bluetooth; controlling the smoking cessation device; updating firmware for the smoking cessation device; displaying data generated by the smoking cessation device; and allowing entry of data and other information, for example by the user, caregiver, or healthcare professional.

In one embodiment, the companion application further comprises functionality chosen from training, coaching, reinforcing, and rewarding the user. Preferably, the companion application resides on a computing device. The computing device may be an essentially non-portable system such as a desktop computer, but preferred computing devices are portable and selected from a list which includes smart phones, tablets, smart watches, laptop computers, smart glasses, and virtual reality systems. In another embodiment, the application resides in the cloud, with functionality such as a user interface and a system for communicating with the smoking cessation device residing on the computing device.

In certain embodiments, a formulation container contains a first formulation, preferably wherein the first formulation is comprised of an active pharmaceutical ingredient such as nicotine. In this embodiment, the formulation container also contains a second formulation, preferably wherein the second formulation does not contain an active pharmaceutical ingredient such as nicotine. In another embodiment, the formulation container contains only a single formulation.

In another embodiment, the smoking cessation system comprises one or more formulation cavities. The formulation cavities may all contain the same formulation, or different formulations, for example, differing amounts and/or concentrations of nicotine or other active pharmaceutical ingredient.

The formulation container can contain at least one formulation chamber, wherein the formulation chamber is a flexible bag housed entirely in the container. Preferably, the formulation chamber is watertight, airtight, and contains a water-vapor barrier to ensure a sealed dosage form. The formulation chamber, may, in one embodiment, be bonded to the inner surface of the formulation container at one or more points, such as, but not limited to, the side of the chamber being bonded to the side of the inner side of a wall of the formulation container, or the top of the formulation chamber being bonded to the inner wall of the top of the formulation container.

The present disclosure provides a formulation container that is part of a component comprising a mouthpiece, hereinafter referred to as the cartridge, wherein this component is disposable and further wherein this component has a through passage that allows for air and aerosol flow to be inhaled by the user, hereinafter referred to as the airway. In one embodiment, the airway may be partly or essentially entirely encircled by the formulation container or by the one or more formulation containers. In another embodiment, the airway is positioned adjacent to the formulation containers, and wherein in a third embodiment the airway is partly or entirely separated from the formulation containers. All of the above-mentioned embodiments have the advantage that the formulation container(s) and other components of the cartridge are changed in a single action. Mouthpiece/formulation container combinations of this type are described in patent application PCT/US2021/064426, incorporated herein by reference in its entirety. Optionally, the airway inlet may reside in the durable part which controls the inhaler, and disposed in the airway section, which is also in the durable part, and is a mechanism that allows for breath actuation of the aerosol. This mechanism is preferably a pressure tap leading to a pressure transducer which is read by a controller in the durable part.

In another embodiment, the cartridge is further comprised of an atomizer. The atomizer may be chosen from a vibrating mesh, a condensation aerosol generator, an ultrasonic nebulizer, a dry powder disperser, a jet nebulizer, a swirl nebulizer, a flow blurring atomizer, and a flow focusing atomizer. Preferably, the atomizer is a vibrating mesh atomizer. Preferably, the atomizer is situated between the airway and at least one formulation container.

The present disclosure also provides that all of the components that contact the formulation or formulations during storage and/or use are contained within the disposable cartridge, and none of the components of the durable system of the aerosolization system are in direct contact with a formulation. It is preferable that the cartridge is designed in such a way to limit the possibility of misuse or abuse of a formulation and concomitant overdose or poisoning. For this reason, it is preferred that the components that are in direct contact with the nicotine, such as mixing chambers, tubing, debubblers, filters, and aerosolizers, are essentially fully contained within the cartridge.

In certain embodiments, the nicotine formulations comprise nicotine or salts of nicotine. Such formulations include, but not limited to, essentially neat nicotine, or nicotine suspensions, or a nicotine solution. One formulation embodiment comprises nicotine and a lipophilic solvent. Lipophilic solvents preferably include an alcohol such as ethanol. Other embodiments include, but are not limited to, a glycol, such as propylene glycol. Another formulation embodiment is a salt of nicotine, preferably nicotine tartrate, more preferably nicotine bitartrate dihydrate, in a solvent comprising water. Alternatively, the formulation is USP nicotine dissolved in a formulation of water or a combination of water and ethanol. The formulation may also comprise various excipients such as surfactants, preferably Tween 20 or Tween 80, stabilizers, absorption enhancers, antimicrobial agents, and the like. In certain embodiments, the formulations comprise a dissolved salt of nicotine and water. In other cases, the formulations consist essentially a carrier, such as water, and a dissolved salt of nicotine (e.g., nicotine bitartrate dihydrate).

In one embodiment, the companion application or the firmware calculates a starting dose of an active ingredient, such as nicotine, which is personalized to the user based on factors chosen from the user's nicotine exposure and smoking history comprising the amount of nicotine per day, cigarettes per day, packs per day, brand of cigarettes smoked, vaping system puffs per day, vaping system used, vaping fluid used, concentration of nicotine in the vaping fluid used, usage of other tobacco products including, but not limited to, pipes, chewing tobacco, and snuff; lifetime cigarette use, for example, as measured by pack-years of smoking; and current craving levels. For example, craving levels can be measured by the Fagerstrom Test for Nicotine Dependence; user physical properties comprising the user's health, age, sex, height, weight, ethnicity, and genetic makeup; previous usage history of other smoking cessation methodologies comprising gums, patches, buccal systems, inhalants, behavioral therapy, and software applications; and the user's usage of the smoking cessation system disclosed herein.

In another embodiment, the starting dose of active ingredient is selected by a physician based on patient characteristics such as weight, smoking history, tolerance to the active ingredient, age, and the like. In a one embodiment, the starting dose of active ingredient is the same for all users, for example, the starting dose of nicotine per puff is about 0.05 mg or more, 0.075 mg or more, 0.1 mg or more, 0.15 mg or more, 0.2, mg or more, 0.25 mg or more, 0.3 mg or more, 0.4 mg or more, or 0.5 mg or more. In one embodiment, the system monitors the inhalation rate during delivery, and turns off the aerosolization if the flow drops below a level that can effectively entrain the aerosol. If the user desires a lower dose, they can remove the mouthpiece from their mouth and continue inhaling, much as is done with a cigarette.

In certain embodiments, the system, for example the device firmware or the companion application, adjusts the amount of an active ingredient, preferably nicotine, delivered per puff, and reduces the amount of nicotine delivered, as the smoking cessation therapy progresses. The adjustment of the amount of active ingredient may be personalized to the subject, based on factors comprising the user's smoking history including, but not limited to, the amount of nicotine per day, cigarettes per day, packs per day, brand of cigarettes smoked, nicotine content of cigarettes smoked, vaping system puffs per day, vaping system used, vaping fluid used, usage of other tobacco products including but not limited to pipes, chewing tobacco, and snuff; lifetime cigarette use, for example as measured by pack-years of smoking; and current craving levels. For example, craving levels may be measured by the Fagerstrom Test for Nicotine Dependence; user physical properties comprising health, age, sex, height, weight, ethnicity, and genetic makeup; previous usage history of other smoking cessation methodologies including, but not limited to, gums, patches, buccal systems, and inhalants, and previous usage history of the smoking cessation system disclosed herein, including but not limited to number of puffs per day, number of dosing events per day, total number of puffs and dosing events, current craving, the reported success not using cigarettes, reported success not using e-cigarettes, past and new health issues, and the rate of change of any of the preceding factors. Preferably, the adjustment of the amount of active ingredient per puff is the same for all users. In one embodiment, the amount of nicotine per puff, when and if the user inhales deeply at or above a predetermined flow rate, is controlled, but the use of the system is otherwise ad libitum, and the user may have as many puffs per dosing event and as many dosing events per day that they may want.

In another embodiment, the dose per puff is adjusted by control of delivered nicotine concentration, which concentration may be adjusted in accordance with an algorithm, wherein this algorithm is used to reduce the dose of nicotine delivered via the smoking cessation system over the course of treatment, wherein the dose of nicotine delivered to the user is, or essentially is, 0 milligrams per milliliter at the end of the treatment. One embodiment of the algorithm predetermines a proper dose level for each treatment, wherein the actual concentration is calculated by the ratio of the historical number of active pulses and the number of sham pulses, wherein the actual concentration is compared to the predetermined concentration, whereupon the pulsing system then pumps a corresponding number of pulses of nicotine containing or non-nicotine containing formulation in order to equilibrate the actual and predetermined concentration. In the above embodiments, the firmware or companion application, as determined by the algorithm, controls a component comprising microfluidic valves, pumps, and the like, wherein this component may be positioned to allow flow from a multiplicity of, preferably two, formulation chambers in order to achieve the proper concentration of nicotine in accordance with the algorithm.

In order to achieve the proper nicotine concentration throughout the treatment, each dose or puff, depending on the embodiment, is given a valve position to modulate the flow of a multiplicity of, preferably two, formulations based upon the comparison of the actual and predetermined nicotine concentration levels of a given day. In one embodiment, a valve is used that partially restricts the flow of each of the multiplicity of formulations in order to achieve the proper nicotine concentration. In another embodiment, a valve is used that entirely blocks the flow of at least one formulation, while allowing for flow of another formulation, wherein there are essentially two valve positions, herein referred to as 0 and 1, wherein each dose or puff, depending on the embodiment, is associated with a 0 or 1, based upon the comparison of the actual and predetermined nicotine concentration levels of a given day, wherein this flow maintains the proper concentration of nicotine in the smoking cessation system throughout the course of the treatment. In one embodiment, there are two microfluidics pumps, wherein each pump pumps from a nicotine containing formulation or a non-nicotine containing formulation, wherein the actuation of the pumps is governed by the aforementioned algorithm. For example, when the actual concentration of nicotine is compared to the predetermined concentration of nicotine for a given day, wherein the actual concentration is higher than the predetermined concentration, the non-nicotine pump will pump formulation for the next pumping cycle. Preferably, this comparison will occur at each dosing event, more preferably, at each puff.

These and other objects, advantages, and features of the invention will become apparent to those persons skilled in the art upon reading the details of the formulations and methodology as more fully described below. Additional embodiments of the present devices, formulations, processes, methods of treatment and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. The patent or application file contains at least one drawing executed in color. Copies of this patent or patent application publication with color drawing(s) will be provided by the USPTO upon request and payment of the necessary fee. Included in the drawings are the following figures:

FIG. 1 is a cross-sectional view of an embodiment of the inhaler device of the present disclosure.

FIG. 2 is a cross-sectional view of an embodiment of the mouthpiece with integrated formulation cavities of the present disclosure.

FIG. 3 is a cross-sectional view of an embodiment of the mouthpiece, aerosolization system, and fluidics system of the present disclosure.

FIG. 4 is a cross-sectional view of an embodiment of the aerosolization system and fluidics system of the present disclosure.

FIG. 5 is a cross-sectional view of an embodiment of the fluidics system of the present disclosure.

FIG. 6 is a cross-sectional view of another embodiment of the fluidics system of the present disclosure.

FIG. 7 is a cross-sectional view of an embodiment of the device of the present disclosure in its entirety.

FIG. 8 is a cross-sectional view of an embodiment of the durable system of the present disclosure.

FIG. 9 is a cross-sectional view of an embodiment of the cartridge of the present disclosure.

FIG. 10a and FIG. 10b are cross-sectional views of an embodiment of a fluid connection point between the cartridge and the durable system body of the present disclosure.

FIG. 11a and FIG. 11b are a cross-sectional views of an alternative embodiment of a fluid connection point between the cartridge and the durable system body of the present disclosure.

FIG. 12 is a cross-sectional view of an embodiment of the cartridge of the present disclosure.

FIG. 13 is a cross-sectional view of an alternate embodiment of the cartridge of the present disclosure with a recirculation system and debubbler.

FIG. 14 is a graph showing the calculated result of a given algorithm for a down titration dosing regimen adjusted each day.

FIG. 15 is a graph showing the error of the down titration dosing regimen of FIG. 14.

FIG. 16 is a graph showing the down titration dosing regimen adjusted for each dosing event.

FIG. 17 is a graph showing the error of the down titration dosing regimen of FIG. 16.

FIG. 18 is a cross-sectional view of an embodiment of the cartridge of the current invention with an integrated filter.

FIG. 19a is a cross-sectional view of an embodiment of a separable pump motor and pump head of the present disclosure, prior to attachment of the cartridge to the durable part.

FIG. 19b is a cross-sectional view of the embodiment of FIG. 19a, during attachment of the cartridge to the durable part.

FIG. 19c is a cross-sectional view of the embodiment of FIG. 19a, after attachment of the cartridge to the durable part.

FIG. 20a is a cross-sectional view of an embodiment of the debubbler of the present disclosure.

FIG. 20b is an isometric view of the debubbler of FIG. 20a.

FIG. 21 is a graph showing the measured emitted dose uniformity of aerosol generated using the embodiment of FIG. 12.

FIG. 22 is a graphical plot of average aerosolized volume over 2 weeks using the embodiment of FIG. 13.

### DETAILED DESCRIPTION OF THE INVENTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art. All publications, patents and patent applications cited herein, whether supra or infra, are hereby incorporated by reference in their entirety.

Where a range of values is provided, it is understood that each intervening value, to the tenth of the unit of the lower limit unless the context clearly dictates otherwise, between the upper and lower limits of that range, is also specifically disclosed. Each smaller range between any stated value or intervening value in a stated range and any other stated or intervening value in that stated range is encompassed within the invention. The upper and lower limits of these smaller ranges may independently be included or excluded in the range, and each range where neither, or both, limits are included in the smaller ranges is also encompassed within the invention, subject to any specifically excluded limit in the stated range. Where the stated range includes one or both of the limits, ranges excluding either or both of those included limits are also included in the invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### A. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

It must be noted that as used herein and in the appended claims, the singular forms “a,” “an,” and “the” include plural referents unless the context clearly dictates otherwise. Thus, for example, reference to “a formulation” includes one or more such formulations and reference to “the method” includes reference to one or more methods and equivalents thereof known to those skilled in the art, and so forth. Further, where the size of an area, such as a hole, is referred to as having a certain diameter, it will be understood that this encompasses other cross-sectional shapes that are not circular but have an area equivalent to a circle of the specified diameter.

About or approximately: In general, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Also, the disclosure of ranges is intended as a continuous range including every value between the minimum and maximum values plus the broadening of the range afforded by the use of the term “about” or “approximately”. Consequently, recitation of ranges of values herein are merely intended to serve as a shorthand method of referring individually to each separate value falling within the range, and each separate value is incorporated into the specification as if it were individually recited herein.

Ad libitum: Dosed according the preference of the user.

Aerosol head cavity: A cavity in the cartridge that contains one or more of air, formulation, an absorbing material, or a debubbler.

Aseptic Filter: A filter used to remove microbes from a formulation. Preferred filters have pore sizes selected from about 0.22 μm, 0.2 μm, or 0.1 μm.

Atomizer: A component of the device of the current invention, preferably of the cartridge, which generates an aerosol. Preferably the atomizer is a vibrating mesh atomizer.

Carrier: A liquid, preferably water, into which an active pharmaceutical ingredient is suspended or preferably dissolved.

Cartridge, disposable, and the like: A component of the device of the current invention with contains the formulation and one or more of a formulation chamber, a formulation cavity, and aerosol head cavity, a debubbler, components of a pump head, tubing, an airway, a mouthpiece, and an aerosolizer. The cartridge is separably attached to a durable system and preferably is generally used for a period of time which is shorter than the period of time the durable system is used for.

Cigarette: An inhalation system that utilizes combustion of tobacco to deliver nicotine.

Condensation aerosol: An aerosol that is formed by first forming a saturated vapor, which vapor condenses to form an aerosol.

Dead volume: The maximum interior volume of a reciprocating pump minus the pump's stroke volume.

Debubbler: A unit for removing air from a formulation, preferably by the use of a hydrophobic porous material.

Dosing event: A series of puffs that are conducted by a user during a single use of a smoking cessation system.

Dry powder dispersing mechanism: A system for adding energy to a packed pharmaceutical powder to form an aerosol.

Durable system: A component which contains one or more of: Electronics, a microcontroller, a battery, a battery charging circuit, a light, a button, an electrical connector, and pump motor, a gear head, an eccentric cam, and an alignment cam.

Eccentric element or cam: An element which is rotated and is not symmetric around the axis of rotation.

e-cigarette: An inhalation system that utilizes electrical power, preferably battery power, to deliver nicotine.

Electronic nicotine delivery systems: An electronic system for smoking cessation therapy or smoking replacement.

Electrospray: A system for forming an aerosol wherein liquid flows out of the end of a capillary and through a hole in a plate. When a voltage is applied between the capillary and the plate, the liquid forms a cone with a jet coming off the tip of the cone. The jet flows through the hole and undergoes Raleigh breakup to form an aerosol.

Emitted Dose: The volume of formulation, or the amount of active pharmaceutical ingredient that is emitted from an inhalation device, for example during 1 puff or one dosing event.

Expanded: A material, preferably a hydrophobic material, which is stretched to make it porous.

Fagerstrom Test for Nicotine Dependence: A test for the severity of nicotine addiction.

Firmware: Computer code that is loaded onto a microcontroller.

Flow blurring: A method of forming an aerosol wherein liquid flows from a capillary and the liquid and a highly pressurized gas co-flow rapidly (for example more rapidly than is used in flow focusing) through a hole in a plate. The liquid at the exit of the capillary is somewhat violently converted to droplets, which are carried through the hole to form an aerosol.

Flow focusing: A method of forming an aerosol wherein liquid flows from a capillary and the liquid and a pressurized gas co-flow through a hole in a plate. The liquid forms a cone under the influence of the co-flowing gas, and a jet is formed at the tip of the cone, which jet undergoes Raleigh breakup to form an aerosol.

Formulation cavity: A void in the cartridge of the current invention that contains the formulation chamber or chambers.

Formulation chamber: A container of formulation for use in the current invention. Preferred formulation chambers are flexible and collapse as formulation is drawn out of them.

Formulation pocket: A region directly behind the aerosolizer which contains formulation, and may also contain a wicking element.

Gauge Pressure: Pressure difference from the surrounding atmosphere.

Jet nebulizer: A system for creating an aerosol from a liquid, wherein a jet of compressed gas is forced through a liquid, forming, entraining, and dispersing the droplets to form an aerosol.

MEMS: A method of fabricating micron sized structures, for example nozzle arrays, using the technology developed for the fabrication of microchips.

Microfluidics: Small pumps, valves, and the like suitable for use in a handheld, portable device.

Nebulizer: A device for creating an aerosol from one or more liquid formulations.

NRT: Nicotine Replacement Therapy.

Orientation agnostic: Capable of being used in any orientations.

Pack years: A measure of the lifetime exposure to cigarettes, calculated as the number of years smoking times the number of packs per day.

PE: Polyethylene

Peristaltic Pump: A device which pumps a formulation through a length of flexible tubing by sequentially compressing portions of the length of tubing.

PET: polyethylene terephthalate

Pharmacologically Equivalent: Having essentially the same pharmacokinetic and pharmacodynamic profile. It is often the case that a pharmacologically equivalent dose can be determined by the ratios of the molecular weights, i.e., having the same number of molecules.

Piezoelectric or Piezo: A component which expands when a voltage is applied to it.

PP: Polypropylene

PS: Polystyrene

PTFE: Polytetrafluoroethylene Puff: A single inhalation containing nicotine.

Raleigh breakup: The formation of an aerosol wherein liquid is forced through one or more nozzle holes to form liquid jets, which jets spontaneously break up, under the influence of surface tension, into droplets with diameters approximately two times the diameter of the jet.

Smoking replacement, nicotine replacement, nicotine replacement therapy, NRT, and the like: A method of reducing the risk of smoking cigarettes by replacing the cigarettes with a more benign form of nicotine, preferably wherein the benign form of nicotine is inhaled.

Smoking cessation therapy, nicotine cessation therapy, and the like: A method of helping tobacco users to quit, preferably by supplying a system that creates an aerosol that contains nicotine, wherein nicotine is gradually tapered down to essentially zero, thereby giving smokers the nicotine they are craving, an experience very similar to smoking, and a pharmacokinetic profile of plasma nicotine essentially identical to that experienced by the smoker after taking a puff from a cigarette.

Sintered: A porous material created through the process of coalescing a powdered material into a porous mass by means of heating without liquefaction.

Stroke volume: The amount of liquid displaced by each cycle of a reciprocating pump.

Subject or patient: Is used interchangeably herein and refers to a human or other mammal.

Substrate: A sheet of material, preferably stainless steel, which contains an array nozzle holes.

Swirl atomizer: A nebulizer that utilizes rotating airflow to form and disperse an aerosol.

Ultrasonic nebulizer: A system for creating an aerosol that applies ultrasonic energy directly to the formulation. Compare to vibrating mesh nebulizer.

Vibrating mesh nebulizer: A system for creating an aerosol that ultrasonically vibrates a substrate which contains an array of nozzle holes.

Wicking element: Means a flexible material that when placed in contact with a liquid will soak up the liquid.

### B. Overview

The present disclosure relates to devices, formulations and methods for inhalation drug delivery. In certain embodiments, the devices are inhalation systems which create an experience to patients that is similar to smoking employing devices with arrays of nozzles for forming a nicotine-containing aerosol. Such inhalation systems control the amount of nicotine delivered to the patient by reducing it essentially to zero over a predetermined amount of time. More particularly, the smoking cessation system of the present disclosure comprises at least one of (a) a portable smoking cessation device; (b) firmware contained in the device for controlling the device; (c) a formulation container; and (d) a companion application.

In certain embodiments, the device is configured as a smoking cessation inhaler, comprising: a durable part (or system or element) controller and a cartridge configured to be separably attached to the inhaler wherein the cartridge comprises (a) an aerosolizer; (b) a formulation chamber containing a formulation comprising an active pharmaceutical ingredient; (c) a debubbler; and (d) a filter. The smoking cessation system is designed to mimic smoking cigarettes by delivering a nicotine-containing composition with nicotine absorption kinetics close to what is experienced with conventional cigarettes and without exogenous excipients/substances as used in e-cigarettes and other devices. Accordingly, the pharmacokinetics (PK), safety profile and user acceptance of the present system is an acceptable alternative to smoking and e-cigarettes and particularly effective for smoking cessation. Like cigarettes, the present aerosol device can mitigate cravings after several doses (or puffs). Yet, unlike smoking and e-cigarettes, the present device does not rely on heat to vaporize the nicotine. Nor does it rely on propellants or other potentially harmful auxiliary or excipient substances. Thus, the compact, hand-held device of the present invention is easy to use, safe and effective in delivering nicotine similar to what a smoker expects.

In one embodiment, the system creates a patient experience that is initially similar to smoking and controls the amount of nicotine administered, reducing it essentially to zero over a predetermined amount of time, for example about 10 to 16 weeks, about 13 weeks, about 20 to 32 weeks, or about 26 weeks, or about 2 months to 4 months, about 3 months, about 5 to 7 months, about 6 months.

More particularly, the present aerosol device is configured to deliver nicotine resulting in pharmacokinetic values that are within about 5%, or 10%, or 15%, or 20%, or 25%, or 30% of those reported in cigarette smokers, wherein the values are one or more of (a) concentration at 5 minutes, 10 minutes and 15 minutes; (b) Cmax; (c) Tmax; and (d) AUC. The reported PK values for cigarettes are stated in the literature. See, e.g., Benowitz N L, Clinical Pharmacology of Nicotine: Implications for Understanding, Preventing and Treating Tobacco Addiction. Clin. Pharmacol. Ther. 83 (4) (2008); Le Nouez, Intl. J. Tuberculosis and Lung Dis. 7(9) (September 2003).

In another aspect, the system of the present invention is configured and dosed in patients to significantly decrease nicotine cravings as measured by the visual analog scale (VAS)-craving assessment. In some embodiments, the present system decreases cravings by about 30%, or 40%, or 50%, or 60%, or 70% compared to baseline.

In certain embodiments, the starting dose of active ingredient is selected by a physician based on patient characteristics such as weight, smoking history, tolerance to the active ingredient, age, and the like. In one embodiment, the starting dose of active ingredient is the same for all users.

For example, upon the initial use of the device, the device may be controlled such that an initial total emitted dose of nicotine free base, or a pharmacologically equivalent amount of another form, for example a salt of nicotine, per average inhalation ranges from about 0.05 to about 2 mg, from about 0.1 to about 1 mg, from about 0.2 to about 0.5 mg, from about 0.25 to about 0.3 mg, whereupon the amount of nicotine will be decreased, for example the flow rate of a nicotine containing formulation will be decreased and the flow rate of a non-nicotine containing formulation increased, such that after a period of time selected from about 1 week to about 1 year, about 1 month to about 6 months, about 2 months to about 4 months, preferably about 3 months, the total emitted dose of nicotine per average inhalation may be approximately 0 mg, and for example wherein the total aerosolized volume of formulation is substantially consistent. This serves as an example only for the purpose of clarification. Preferred starting amounts of nicotine per puff are preferably greater than or about 2 mg/puff, greater than or about 1.5 mg/puff, greater than or about 1.2 mg/puff, greater than or about 1 mg/puff, greater than or about 0.8 mg/puff, greater than or about 0.6 mg/puff, greater than or about 0.5 mg/puff, greater than or about 0.4 mg/puff, or greater than or about 0.3 mg/puff. Ending amounts of nicotine are preferably less than or about 1 mg/puff, less than or about 0.8 mg/puff, less than or about 0.7 mg/puff, less than or about 0.6 mg/puff, less than or about 0.5 mg/puff, less than or about 0.4 mg/puff, less than or about 0.3 mg/puff, less than or about 0.2 mg/puff, less than or about 0.1 mg/puff, or about 0 mg/puff.

### C. Aerosol Device and its Operation

As described above, the present disclosure relates, in certain embodiments, to a smoking cessation inhaler, comprising: a durable part (or system or element) of the inhaler comprising a controller and a multidose cartridge configured to be separably attached to the durable part of the inhaler wherein the cartridge comprises (a) an aerosolizer; (b) a formulation chamber containing a formulation comprising an active pharmaceutical ingredient; (c) a debubbler; and (d) a filter.

In one embodiment, the cartridge comprises one drug chamber, and the dose is controlled by one or more mechanisms chosen from, but not limited to, a pump, a pumping time, a valve, a valve open time, value of a DC voltage applied to an aerosolizer, the amplitude of an AC voltage applied to an aerosolizer, and a time duration of the application of a voltage, for example a voltage that is applied to an aerosolizer.

In one embodiment, the algorithm calls for the adjustment of the amount of active ingredient, preferably nicotine, delivered by the system at a frequency selected from the list comprising but not limited to about once per day or greater, about once per dosing event or greater, or about once per puff. Preferably the algorithm calls for the adjustment of the of the amount of active ingredient about once per day, preferably before the first puff of the day. Preferred algorithms change the dose based on a parameter selected from the list comprising but not limited to the number of puffs since the first puff, number of dosing events since the first dosing event, time from the first puff, time from the first dose, days since the first day, a combination thereof. Preferred algorithms selected from the list comprising but not limited to a linear increase or decrease with the parameter, a quadratic increase with the parameter, a higher order polynomial with the parameter, an exponential increase or decrease with the parameter. Preferably, the algorithm calls for the change of the amount of active ingredient from an initial value to a final value over a time period selected from the list comprising but not limited to about 1 day to about 1 year, about 1 week to about 9 months, about 1 month to about 6 months, about 2 months to about 4 months, or about 3 months. Preferably the algorithm calls for an increase in the amount active delivered, a decrease in the amount of active delivered, or both. In one preferred embodiment, the algorithm calls for a decrease from one dose, preferably a starting dose, to a final dose, preferably about zero. In another preferred embodiment, the algorithm calls for an increase from an initial dose at a first event, preferably a starting day, to a second dose at a second event, preferably a later day, a constant dose from the second event to a third event, preferably a still later day, and a decrease from the second dose to a final dose at a fourth event, wherein the final dose is preferably zero. Preferred events are selected from the list comprising but not limited to days, dosing events, and puffs.

The smoking cessation system, preferably one or more of the smoking cessation device, firmware, and or companion application, is preferably comprised of a way of monitoring, recording, displaying, and/or reporting various factors related to the usage of the smoking cessation system, selected from the list including but not limited to the time, date, frequency, and/or total number of puffs and/or the number of dosing events and/or rate of change of any of the preceding. The system may also monitor, record, report, and/or display parameters related to the usage technique of the system, including but not limited to depth of inhalation, rate of inhalation, and coordination of a button push with the inhalation maneuver. Reporting is selected from a list including but not limited to reporting to a user, physician, nurse, physician group, HMO, insurance company, research organization for example a clinical research organization, investigator, epidemiologist, manufacturer, help line, care giver, family member, and/or friend. Reporting may require opt in by the user, and reporting, data storage, and data transmission are preferably HIPAA compliant.

In one embodiment, there may be a one-way data flow from the smoking cessation device to the companion application. In this embodiment, the companion application may perform functions selected from a list including but not limited to displaying information, reporting information, for example to a health care provider, care giver, family member, and/or to the supplier of the smoking cessation system. The information transmitted to the companion application may include information selected from the list including but not limited to the time, date, and number of puffs and/or dosing events, current amount of an active ingredient, preferably nicotine, per puff, battery status, remaining formulation, error codes, firmware revision, and the like.

In another embodiment, there may be a two-way data flow that allows the companion application to communicate with and/or control the smoking cessation device. In a preferred embodiment, the companion application will calculate and instruct the smoking cessation device as to a formulation flow rate, preferably the flow rates of a first and a second formulation, thereby controlling the amount of an active ingredient, for example the amount of nicotine per puff, delivered, as described previously. The companion application may also instruct the smoking cessation device to turn on, to ready itself for a change of formulation container, to display an indicator, for example a ready indicator, to utilize a given rate of flow of a formulation, preferably 2 or more formulations.

In one embodiment, the firmware or the companion application may incorporate a timing system, and be programmed for, or may calculate, a lockout interval before the next puff and/or dosing event. The lockout interval may be constant for the duration of the therapy, for example the smoking cessation therapy, but in a preferred embodiment the lockout interval is increased over time. The initial lockout interval, and/or the rate of increase of the lockout interval, may be fixed by programming of the companion application or the firmware, but is preferably calculated by the companion application or firmware based on previously entered information related to, for example, the user, the users smoking history, and the previous usage of the smoking cessation system, as described above. Preferably the smoking cessation system incorporates one or more indications that the lockout interval has elapsed. Preferred indications include but are not limited to a light, a message on a display, a notification, a sound, and/or a vibration. For some applications, for example the delivery of opioids, the lockout interval may be prescribed by the prescribing physician.

Preferably, the smoking cessation device incorporates a mechanism for forming an aerosol from one or more formulations, hereinafter referred to as the aerosolizer. The aerosolizer may be selected from the list including but not limited to a vibrating orifice or array of orifices (i.e., a vibrating mesh), a condensation aerosol, an ultrasonic nebulizer, a dry powder dispersing mechanism, a jet nebulizer, a swirl atomizer, Raleigh breakup, an electrospray, flow focusing, or flow blurring. In a preferred embodiment, a fine aerosol is generated from one, preferably a plurality, of small holes. In a particularly preferred embodiment, the aerosolizer is comprised of an array of nozzle holes formed in an essentially rigid substrate, preferably a stainless steel substrate, and the substrate is oscillated by a ring-shaped piezo element that is rigidly bonded to the perimeter of the substrate on at least one side, wherein the array of holes is substantially centered in the opening of the ring-shaped piezo element. The substrate may be comprised of a protrusion which may be in the shape of a section of a sphere, protruding from the exit side, which protrusion is substantially centered in the middle of the ring-shaped piezo element and have the array substantially centered on the protrusion. The protrusion extends toward an airway through with the user inhales, and preferably the protrusion extends into the airflow through the airway.

The piezo may be attached to the exit face of the substrate, or there may be two piezos, one attached to each face of the substrate. A preferred embodiment has a single piezo ring attached to the entrance face of the substrate, which removes the step from the piezo to the center of the ring from the airway where such a step can cause aerosol deposition. In a preferred embodiment, the substrate is a disk and the piezo is an annulus. When liquid is supplied to the entrance side of the array and the substrate is oscillated by the piezo, an aerosol is formed at the exit-side of the array. In one embodiment, the amplitude of the oscillation is such that a single droplet is forced out of each nozzle hole with each oscillation cycle of the piezo. Alternatively, a length of liquid jet may be extruded from the exit nozzle of each hole, which length of liquid jet breaks up into droplets via the process of Raleigh breakup. Preferably there is a one or more holes with an exit diameter which is less than or about 10 μm, preferably less than or about 7.5 μm, less than or about 6 μm, less than or about 5 μm, less than or about 4 μm, less than or about 3 μm, less than or about 2.5 μm, less than or about 2 μm, less than or about 1.5 μm, less than or about 1 μm preferably between about 2.0 and 3.0 μm, between about 2.2 and 2.8 μm, most preferably about 2.5 μm. Preferably there are less than about 100,000 holes, between about 1 and about 10,000 holes, between about 100 and about 5000 holes, between about 500 and about 3000 holes, or between about 750 and about 2500, more preferably between about 2000 and 2400 holes. The channel leading to the exit of the orifice(s) may be any shape, including a cylinder, preferably a right circular cylinder. Preferred channels are tapered, decreasing in diameter from the entrance of the orifice to the exit, in order to reduce the pressure or other form of energy input, for example ultrasonic energy input, required to achieve flow through the orifice and aerosolization. Preferably, the taper is chosen from a cone shape, a pyramidal shape, and funnel shape. Preferably the inlet side of the orifices is greater than or about 5 μm, greater than or about 10 μm, greater than or about 15 μm, greater than or about 20 μm, or greater than or about 25 μm. The array of holes can be fabricated using a method selected from the list including but not limited to laser drilling including but not limited to UV lasers, excimer lasers, solid state lasers, diode pumped lasers, frequency doubled lasers, frequency tripled lasers; MEMS fabrication, molding, machining, 3D printing, stereo lithography, water jet, wire EDM, and the like. A surface treatment may be applied to the exit side of the array to improve aerosolization performance. By way of example, if the formulation is an aqueous formulation, then a hydrophobic treatment may improve aerosol performance. In another embodiment, a hydrophobic treatment may be applied to the entrance side of the array in order to prevent liquid egress out of the apertures in the array. This hydrophobic treatment, may, in one embodiment, be tailored to prevent liquid egress out of the apertures at atmospheric pressure, within a range of pressures, or up to a specific pressure. Preferred treatments include plasma treatment, silicone, fluorine-based liquids or polymers, and waxes.

In a preferred embodiment, the aerosolizer is contained within the aerosolization head, wherein the aerosolization head comprises the aerosolizer, a pocket directly behind the aerosolizer holding a formulation or a mixture of formulations and an entry port for formulation to be introduced into the pocket. Preferably the aerosolization head, one or more formulation chambers containing one or more formulations, and a cavity that surrounds the formulation chamber or chambers are integrated into the cartridge. In a preferred embodiment there is a formulation pocket in the aerosolization head directly behind the nozzle array. Preferably this pocket is substantially filled with a formulation or mixture of formulations, and the formulation is in contact with the back of the substrate in the area of the nozzle array. In a preferred embodiment the aerosolizer, on the orifice entrance side, is in direct fluid contact with the formulation or mixture of formulations in the pocket. The formulation pocket may also contain air, including air introduced into the pocket as a byproduct of the aerosolization process. It is important that the air in the pocket be removed so that it does not displace the formulation in contact with the substrate. In another embodiment, formulation is fed to the entrance side of the array via a wicking element that is located within the pocket, wherein this wicking element may be, but is not limited to, a sponge; a foam, which may be an open cell foam or a closed cell foam, preferably a combination; a fabric, a thread, yarn, filament or the like; an absorbent paper product; or the like. Preferably the wicking element is in contact with the entrance-side of the substrate, or partially in contact with the entrance-side of the substrate. In one embodiment the wicking element may be flush against the entrance side of the array of nozzle holes. In another embodiment the wicking element may be positioned at an angle, such that the wicking element is in contact with only part of the array of nozzle holes, or none of the array of nozzle holes, wherein a small space between the wicking element and the array of nozzle holes becomes filled with liquid by the pumping action of the nozzle holes and serves to supply formulation to the nozzle holes. In yet another embodiment, a small bump, or a multiplicity of bumps, may be created in the wicking element, which bumps are positioned directly behind the array of nozzle holes, whereupon the array of nozzle holes come into proximity or contact with the bumps in the wicking element, creating an uneven surface contact between the wicking element and the entrance-side of the aerosolizer. In another embodiment, the wicking element is separated from direct contact with the entrance side of the nozzle array by a porous spacing element that allows for flow of formulation and unimpeded flow of air to facilitate the removal of the air. The spacing element may be a mesh, a plate with an array of through holes, a spacer with a single hole substantially the same size as the array, or by a foam with greater porosity and/or larger pores than the wicking element, wherein this element allows for formulation to flow from the wicking element to the entrance-side of the array of nozzles and simultaneously for the clearance of air that is generated and introduced into the aerosolization head from the aerosolizer. In one embodiment, the wicking element and the spacing element have flat surfaces that are circular, or substantially circular, and have the same diameter as the hole at the center of the piezo annulus. In another embodiment the wicking element may be either larger of smaller in diameter than each other, and either larger or smaller in diameter than the diameter of the center of the piezo annulus. In another embodiment the wicking element may be of substantially the same diameter and in contact with the dimple.

In one embodiment, the pocket, and similarly the formulation cavity or container is constructed of a relatively hard material, such as a plastic or metal material, including but not limited to PE, PS, PP, PET, stainless steel, aluminum and the like, and may be entirely enclosed other than a path for ingress, and optionally a path for egress, of formulation. In another embodiment the formulation pocket may contain at least one air release opening that allows for flow of air that is generated in the formulation pocket out of the formulation pocket and into an aerosol head cavity. As air released through the air release opening accumulates in the aerosol head cavity, it may approach or exceed the pressure of the formulation in the region of the air release opening, reducing or stopping the flow of air through the air release opening. Thus, there needs to be a vent to release the air from the aerosol head cavity. In one embodiment the air is vented from the aerosol head cavity through a vent hole to the air outside the device. In another embodiment the air is vented through a vent hole leading into the formulation cavity, the flow through which is assisted by the decrease in pressure in the formulation cavity that arises as formulation is pumped out of one or more formation chambers in the formulation cavity. Because the rate of generation of air and the rate of aerosolization of formulation are in general not equal, a small hole may be used to create contact with the outside air. Preferably the small hole is sized such that equilibration with the outside air requires more time than the time to aerosolize and remove air from the formulation pocket. Preferably the small hole is also small enough that the diffusion of water vapor through the air release opening is small enough that the rate of evaporation of water into the aerosol head cavity saturates the air in the aerosol head cavity, reducing the rate of evaporation of the formulation carrier.

Preferably the air release opening is a small, optionally a tapered, opening, and the inside surfaces of the aerosol release opening are hydrophobic, such that air is able to flow from the pocket through the air release opening, but the surface tension of water prevents, or substantially prevents, the flow of liquid formulation through the opening. In one embodiment there may be only one opening, preferably located at the top of the top, or in an alternative embodiment on the side of the formulation pocket, wherein air may reside in the formulation pocket until it is able to exit the chamber under the force of gravity as it is separated from the formulation due to the force of buoyancy. In another embodiment the formulation pocket may contain a one or more openings, wherein air is able to immediately exit the pocket under its buoyancy force through one or multiple of the openings, preventing air buildup in the pocket in any orientation and rendering the smoking cessation system orientation agnostic.

In a preferred embodiment, an air release opening may be covered with a porous material, preferably a hydrophobic or superhydrophobic porous material, wherein the porous hydrophobic material allows for air to exit through the opening and prevents, or substantially prevents the exit of formulation through the opening. This porous material may be, but is not limited to, a metal mesh, a plastic mesh, or, in a preferred embodiment, a PFTE or PE material, preferably an expanded or sintered PTFE or PE material. A hydrophobic coating or process may be applied to these materials to enhance their hydrophobicity. The opening may be a large area, for example comprising a wall of or essentially the entire surface of the formulation pocket not closed by the aerosolizer. In this embodiment the porous material that comprises the formulation pocket may allow air to exit from the formulation pocket at any point along the porous material, preventing the creation of an air-filled headspace within the formulation pocket.

In a preferred embodiment the cartridge may contain a system for recirculating formulation or mixture of formulations, hereinafter referred to as a recirculation system. In a preferred embodiment a first fluid passageway extends from the formulation chamber to a pumping element or one or more pumping elements, a second fluid passageway extends from the pumping element to the pocket, and a third fluid passageway extends from the pocket to the first fluid passageway, creating a flow loop that enables recirculation. These passageways may be, but are not limited to, tubes, channels, or mixtures thereof. In a preferred embodiment, situated in or at an end of one or more the passageways is a debubbler, wherein the flow through the one or more passageways flows through the one or more debubblers. In a preferred embodiment the debubbler comprises at least one debubbler channel configured to allow flow through the debubbler, preferably a multiplicity of the channels, preferably wherein the channels are narrow channels, wherein at least one surface of each channel is formed by a porous material and flow through the debubbler brings the formulation in the recirculation system into contact with the porous material upon which the air is expelled out of the porous material. Preferably the porous material is as described above, most preferably an expanded or sintered PTFE or PE material. In a preferred embodiment there is a restriction in the passageway downstream from the debubbler, wherein this restriction increases the pressure within the debubbler to increase the amount or rate of air clearance through the porous material.

In a preferred embodiment, the porous material is held taut against a surface opposite the porous material by means of compression. In another embodiment the porous material may be held taut against the opposite surface by means including, but not limited to, gluing, taping, chemically binding, or mechanically clamping. In the process of clamping, the porous material may be stretched beyond its natural state. In this embodiment, when formulation flows under the porous material at a pressure above atmospheric pressure it deforms the porous material away from the opposing surface. This creates a small cavity behind the porous material for liquid flow. When the formulation stops flowing through the debubbler the porous material rebounds to its starting position, reducing or eliminating the volume of the small cavity and pushing the formulation out of the exit and/or entrance of the debubbler, minimizing the amount of formulation that may remain in contact with the porous material, thereby limiting the amount of carrier that can evaporate through the porous material. In one embodiment the air that is expelled through the porous material is expelled out of the cartridge, wherein this may be accomplished by having the porous material positioned on the exterior surface of the cartridge. In another embodiment air exits the cartridge via a passageway connecting the debubbler to the surface of the aerosolization head. In another embodiment the air that is expelled from the debubbler flows into the formulation cavity, wherein the reduced pressure that is created in the formulation cavity by formulation being pumped out of the formulations chamber(s) pulls the air from the debubbler, thereby restoring the pressure in the formulation cavity substantially to atmospheric pressure. In a particularly preferred embodiment, there is a chamber attached to the back of the debubbler, wherein the air that exits from the porous material of the debubbler enters the chamber. The chamber may be a plastic molded chamber with a multiplicity of, or preferably one, hole, wherein this hole is preferably less than about 5 mm in diameter, more preferably less than about 4 mm, more preferably less than about 3 mm, more preferably less about than 2 mm, more preferably less than about 1 mm, more preferably less than about 0.5 mm, and most preferably less than about 0.1 mm in diameter (or the equivalent area if the hole is not a circle). This allows the pressure increase from the air that exits from the debubbler to be released out of the hole, but limits evaporation of formulation carrier through the porous material of the debubbler between dosing events. Preferably the rate of evaporation from the system from all sources (including the debubbler and the nozzle array) is less than or about 20 μl/day, less than or about 10 μl/day, less than or about 5 μl/day, less than or about 2.5 μl/day, or less than or about 1 μl/day. In one embodiment the recirculation system may additionally contain an aseptic filter, preferably a filter with a pore size of about 0.1 to about 0.5 μm, about 0.15 to about 0.3 μm, preferably about 0.2 μm, to remove particulates and bacteria from the formulation. In a preferred embodiment the aseptic filter is located in the second channel, between the pump and the formulation pocket, in order that the high pressure required to force formulation through the aseptic filter is not present in the pocket and therefore does not force formulation out of the nozzle holes. This design ensures that the formulation that is aerosolized and inhaled by the user is made free of bacteria and particles that might pose a danger to the user and also might block nozzle holes. The aseptic filter is preferably a filter that is designed to be used for aseptic filling of aqueous formulations in the medical industry. The aseptic filter may be hydrophobic or hydrophilic, wherein a preferred embodiment would use a hydrophilic membrane.

In a related embodiment, the debubbler is comprised of a small debubbler cavity, which cavity has a small hole as described above, leading to the aerosol head cavity or the formulation cavity, thereby limiting the rate of evaporation of formulation carrier through the debubbler. Preferably the small hole of the embodiments above is less than or about 2 mm in diameter, less than or about 1.5 mm in diameter, less than or about 1 mm in diameter, less than or about 0.75 mm in diameter, less than or about 0.6 mm in diameter, less than or about 0.5 mm in diameter, less than or about 0.4 mm in diameter, less than or about 0.3 mm in diameter, or less than or about 0.2 mm in diameter.

In one embodiment, at least one of the formulation chambers is flexible and maintained in liquid contact with the recirculation system. If there is a reduction in surrounding air pressure, for example due to weather or descent from altitude, the expansion of any air bubble in the recirculation system will expand the formulation chamber rather than forcing liquid out of the nozzle. The expandable formulation chamber may be left open to the recirculation system at all times or may be opened and closed under the control of an electronically enabled valve in order to properly regulate the pressure of the recirculation system by allowing for flow into and out of the formulation chamber to keep the system at, or approximately at, atmospheric pressure as it experiences pressure changes in the external environment, altitude, or changes in orientation.

The formulation cavity within the cartridge may be, in one embodiment, an enclosed partially air-filled volume. In a preferred embodiment there may be at least one connection point between the pocket and the formulation cavity, wherein air may pass through the connection point. In one embodiment, wherein the pocket and the formulation cavity are adjacent, the opening may be a section of shared wall that is removed to facilitate this air passage. This opening may, in one embodiment, be covered with a porous material, preferably the porous material as described above, that allows for the flow of air through the material but blocks the flow of formulation. In another embodiment, wherein the cavity and formulation container(s) do not share a common wall, the opening may be a passageway, preferably a tube, more preferably a machined channel, that allows for air flow. In one embodiment this passageway may lead to a larger opening in the wall of the formulation cavity which is covered with the porous medium as described above. In the above embodiments the formulation cavity may be an air-filled cavity.

In another embodiment, the aerosol head contains an aerosol head cavity, adjacent to and optionally partially surrounding the formulation pocket. In one embodiment the aerosol head cavity may also contain an absorbent material, which may be, but is not limited to, a sponge, foam, cloth, paper, or other absorbent material. In this embodiment any formulation that does exit from the formulation pocket will be absorbed into the absorbent material. As the smoking cessation system is used and an aerosol is generated, formulation is pumped out of the formulation chamber(s) creating a reduced pressure within the formulation cavity. This reduced pressure will pull air from the aerosol head cavity through a channel that connect the two components, reducing the air in the aerosol head cavity. This will encourage the flow of air, for example air generated by the aerosolization process, from the aerosol pocket into the aerosol head cavity, preferably through a porous hydrophobic material. The amount of air generated into the formulation pocket during the aerosolization process may be equivalent to or less than the amount of formulation that is pumped out of the formulation chamber(s), wherein this will allow for all of, or substantially all of, the air within the formulation pocket to exit out of the formulation pocket into the aerosol head cavity, wherein the cavity will return to atmospheric pressure or retain a slightly reduced pressure.

The aerosolizer is positioned such that aerosol is generated into the device airway. For example, in the embodiment wherein the aerosolizer is a vibrating mesh aerosolizer, the side of the substrate that contains the exit orifice(s), herein referred to as the exit side, is flush, or substantially flush, with a wall of the airway. When actuated, the aerosolizer creates an aerosol plume that travels into the airway perpendicular to the direction of the air flow that is generated by the user, herein referred to as the dilution air, wherein the dilution air entrains the aerosol at a 90° angle and carries into the user's respiratory tract. The user inhales through this airway via a mouthpiece that extends from the end of the airway, wherein this mouthpiece may be a hollow or partially hollow cavity of varying lengths, shapes, and sizes. Preferably at least a section of the airway, and also the mouthpiece, are part of the cartridge.

The airway in the cartridge originates upstream from the point of aerosol generation and ends at the tip of the mouthpiece that is inserted into the user's mouth, downstream from the point of aerosol generation. The airway may be curved upstream from the point of aerosol generation and straight at and downstream from the point of aerosol generation, more preferably being straight or substantially straight throughout the length of the airway. In another embodiment the centerline of the airway may be substantially straight downstream from the point of aerosol generation, and the walls may have a slight divergent angle, preferably the walls diverge from the centerline at an angle of less than or about 7° beginning at a point downstream of the point of aerosol generation and continuing as far as to the tip of the mouthpiece that is inserted into the user's mouth. The interior walls of the airway may, in one embodiment, contain an arrangement of extrusions and grooves, running either vertically, horizontally, or a combination of both, such that they create a purposeful turbulence that is intended to separate aerosol particles before they agglomerate. In a preferred embodiment the inside walls of the airway are smooth or substantially smooth to allow for uniform flow.

To prevent evaporation of formulation from the aerosolization head, the formulation pocket is preferably substantially completely sealed in the aerosolization head, with the only openings being one or more formulation feed lines leading to the formulation chamber(s), the array of nozzle holes, and, in various embodiments, the opening(s) to allow for flow out of the formulation pocket, and/or the micropores in the porous material used to allow air to exit from the formulation pocket. In one embodiment, in order to limit evaporation of formulation carrier through the nozzle holes and also to limit drying of formulation and creation of solids from formulation deposited at the exit side of the nozzle, the exit side of the array is sealed between doses by a sealing system that can be removably positioned in a first position to isolate the nozzles from the air outside of the device when between doses or puffs, and can be moved to a second position when the user is inhaling through the airway. When the sealing system is in the first position between uses of the device, it will substantially seal a region around the exit of the nozzle array to maintain that region as essentially 100% relative humidity (due to evaporation of carrier from formulation that has deposited outside of the nozzles) and thereby keep the wicking element or formulation pocket from losing carrier and increasing concentration due to diffusion of formulation carrier through the nozzle holes. In addition, any formulation coating the exit side of the substrate after aerosol generation cannot evaporate in the high relative humidity environment, and therefore cannot leave a layer of dried formulation components that can disrupt subsequent aerosol generation. The closing of the sealing system in the first position can be accomplished by an element that blocks the air into and out of all of or a section of the airway. In a related embodiment, the sealing is accomplished by a moveable element that blocks the entrance to the airway in combination with a mouthpiece cover that is replaced by the user after dosing In this embodiment the region of the airway that is sealed off from the surrounding air is an air-tight, or substantially air-tight, air-filled space, wherein aerosol droplets deposited by the preceding dosing event are on the airway walls due to mechanisms including turbulent deposition, inertial impaction, interception, and gravitational settling. In a preferred embodiment, the aerosol sealing system is optimized such that the amount of formulation deposited on the walls is more than enough or essentially just enough to saturate the air contained in the sealed off section of the airway with evaporated carrier, inhibiting further evaporation of carrier or drying of the formulation that is in, on, or around the nozzles or the exit side of the array of nozzles. In another embodiment the sealing system comprises a lever arm or the like with a compliant material on one end, which compliant material may be, but is not limited to, silicone, rubber, PTFE, a closed cell foam, or another flexible material, whereupon the material is in contact with the substrate and blocks all or substantially all of the nozzles of the array when in the first position, to prevent formulation leakage, evaporation, and drying. In another embodiment, an additional material which is attached to the compliant material and is in contact with the nozzles and is absorbent, and absorbs any formulation deposited on the substrate. In one embodiment, the movement of the sealing system from the first to the second position is accomplished by the pressure of a patient inhaling through the airway. In another embodiment the movement of the sealing system is accomplished by means of an electromechanical actuator powered by a system battery and controlled by a microcontroller. Preferably, the opening and closing of the sealing system is actuated by the force of the user pressing a button, which preferably also readies the device for delivery and/or triggers the start of aerosol generation. In one embodiment this may be a mechanical system, wherein the moving of the sealing mechanism to the second position is powered by the physical force of the user pushing the button. In another embodiment, the moving is accomplished by an electro-mechanical system that is triggered by the user pressing the button. In one embodiment, the sealing system is returned to the first position by an electro-mechanical actuator. In another embodiment, the system is returned to the first position by a return spring or the like. In one embodiment, aerosol generation is stopped when the user releases the button, or when a predetermined amount of formulation has been aerosolized, whichever comes first, and the sealing system is returned to its position sealing the nozzle array after a predetermined delay to allow the user to inhale the aerosol deep into their lungs.

In one embodiment the wicking element of the formulation pocket absorbs any liquid that remains on, in, or around the nozzles after aerosolization has ended. This will prevent liquid from drying in and around the nozzles, which might otherwise result in dried formulation components and disrupt future aerosolization. The wicking element may accomplish this in any orientation, including being positioned entirely or partly flush, or angled, against the entrance-side of the array of nozzles on the aerosolizer. In another embodiment a reduced pressure may be created within the aerosolization head, or components of the aerosolization head, such that, at the end of aerosolization, the pressure in the formulation pocket is below atmospheric pressure, whereupon air will flow into the formulation pocket of the aerosolization head through a nozzle or nozzles, and may suck in any formulation that is in, on, or around the nozzles after aerosolization has ended. This may be accomplished, for example, by running a pumping element backward, or by limiting the rate at which air can flow into the formulation cavity, resulting in reduced pressure and expansion of the aerosol cavity after aerosolization.

In a preferred embodiment, the aerosol is generated into an airway at an essentially 90° angle to the direction of dilution air flow through the airway. In this embodiment, the dilution air flow, as generated by the user inhaling, entrains the aerosol at a 90° angle and carries it out of the airway and into the user's mouth and lungs via the mouthpiece. In a preferred embodiment the airflow through the airway is laminar, or substantially laminar, flow. To reduce the amount of aerosol deposited on the airway walls a preferred embodiment contains a pressure sensor that detects a pressure drop in the airway when the user is inhaling, whereupon when the controller determines that a predetermined pressure drop has been exceeded, it actuates the aerosolizer and thus the generation of aerosol. This ensures that aerosol is only generated when there is a sufficient amount of dilution air flowing through the airway to entrain the aerosol and carry it into the user's mouth and lungs. In a preferred embodiment the aerosol is generated for a fixed amount of time unless the user's inhalation rate drops below a threshold value while the aerosol is being generated, in which case the controller senses the lack of sufficient inhalation airflow rate and stops the aerosolization. In one embodiment, the controller calculates an inhalation flow rate based on previous calibration of the measured pressure drop, and calculates an inhaled volume, for example by integrating the inhalation flow rate over time. In this embodiment, the controller actuates the aerosolizer if both the fixed amount of time has not been reached, and the controller estimates by way of a predetermined algorithm that the user has sufficient remaining lung volume to inhale the aerosol deep into their lungs. In one embodiment, if the aerosol generated was stopped before the fixed amount of time was reached because the measured pressure dropped below the predetermined threshold, the controller will re-start aerosol generation if the user increases their inhalation rate and again exceeds the pressure threshold while still having sufficient lung volume remaining. In the case that the target fixed amount of aerosolization time is not achieved, an amount of formulation delivered to the aerosolization head via the pumping system is reduced accordingly to maintain an ideal pressure within the formulation pocket without the presence of an air-filled headspace. To guide the user to inhale at the correct flow rate, the device may present a signal, such as a sound, vibration, or preferably a light when an adequate flow rate is achieved. In one embodiment, the user is presented with a steady light, for example a green or blue light, when the user is inhaling withing a predetermined optimal range of flow rates, no light when the user inhaling at a flow rate below the optimal range, and a different, for example red and/or flashing light, when the inhalation flow rate is above the optimal range.

The pressure in the formulation pocket may be monitored via a pressure transducer, wherein the pumping system delivers formulation to the formulation pocket to maintain it at or just below the ideal pressure. In one embodiment, the ideal pressure is less than or about 100 mm Hg, less than or about 50 mm Hg, less than or about 25 mm Hg, less than or about 10 mm Hg, less than or about 7.5 mm Hg, less than or about 5 mm Hg, less than or about 2.5 mm Hg. Preferably the pressure is substantially zero. Zero pressure is preferred to eliminate liquid being forced through the nozzle array. In one embodiment, a valve is placed close to the outlet of the formulation chamber. When the pressure transducer senses gauge pressure above zero (pressure in the pocket is above the pressure of surround air), the valve is opened, allowing the pressure to be alleviated by liquid flowing into the flexible formulation chamber. When the pressure transducer senses that gauge pressure is below zero, (pressure of the surrounding air is above the pressure in the pocket) the valve is closed, preventing air from being forced into the nozzle holes as the flexible formulation chamber expands.

Preferably, the mouthpiece is rigidly attached to the cartridge housing, more preferably is a feature that is machined, preferably molded, as a part of the cartridge housing. Preferably the atomizing element is configured such that the piezo ring is attached to substrate on the side of the substrate away from the airway, and the perimeter of the substrate is flush with the airway wall, to prevent a step which can result in non-laminar flow and aerosol deposition in the airway. Preferably the aerosolization head contains a structure that prevents the aerosolizer from becoming dislodged or falling into the airway. Preferably the aerosolizer is not rigidly bonded to the aerosolization head, rather the aerosolizer is bonded to the aerosolization head by a process or material, including but not limited to silicone adhesion, wherein this bonding prevents, or substantially reduces, damping of the oscillations of the piezoelectric transducer in order to avoid a deleterious impact on aerosolization rate and quality. In a preferred embodiment, the adhesive, preferably silicone, also serves to seal the piezo and prevent the ingress of carrier from the formulation into the piezo.

The above-mentioned components, the mouthpiece, airway, aerosolization head, pocket, formulation cavity, channels, tubing, and formulation chambers(s), may be contained in one component referred to as the cartridge. The cartridge comprises four primary components; namely the mouthpiece, the airway, the aerosolization head and its sub-components, and the formulation containment and delivery system and its sub-components. Other components, including battery, pressure transducers, pump motors, microcontroller, button(s), indicator lights, displays, and other electronic components are contained within the durable system element part of the system, and the cartridge is removably attached to the durable system element.

In a preferred embodiment, the durable system contains a system for moving liquid formulation from the formulation chamber(s) in the cartridge to the formulation pocket of the aerosolization head, preferably a system for pumping the formulation at controlled flow rates, wherein this system may comprise one or more pumping mechanisms or other suitable systems for moving a liquid formulation, hereinafter referred to as the pumping system and the pump(s), respectively. Preferably, the rate of flow of each formulation, the flow of which may occur simultaneously and/or sequentially with aerosol generation, is determined by a computing system, wherein the computing system may either be located within the device or located remotely, for example within a mobile application that performs the necessary calculations to determine the proper flow rates. Furthermore, the pumping system may be intended to mix a one or more formulations into one formulation, wherein this mixed formulation is delivered to the formulation pocket within the aerosolization head. In one embodiment, the flow of the formulations is accomplished by a method including but not limited to the use of two separate microfluidic pumps, wherein one microfluidic pump moves an active ingredient, preferably nicotine, containing formulation, and one microfluidic pump moves a second, preferably non-active ingredient containing formulation. In a preferred embodiment, this pumping system uses the microfluidic pumps and tubing to create a fluid connection between the formulation container and the entry port to the formulation chamber of the aerosolization head.

In one embodiment, the pumping system is comprised of a mixing system for mixing a multiplicity of, preferably two, formulations prior to aerosolization. In one embodiment, the sum of the volumes of the first and second formulations introduced into the mixing system always sum to the same amount. This amount may be the volume of formulation aerosolized by the aerosolization system per each puff, or per a fixed number of puffs, for example 2 or more, 3 or more, 4 or more, 5 or more, 6 or more 8 or more, 10 or more, 15 or more, or 20 or more. In a preferred embodiment, the sum of two volumes is variable, and the volume may be selected from the list including but not limited to: an expected amount to be aerosolized, an expected amount to be aerosolized per dosing event, an expected amount to be aerosolized per puff, an amount previously aerosolized, an amount previously aerosolized in a dosing event, and an amount previously aerosolized in a puff.

In one preferred embodiment, the pumping system contains a mixing system, wherein the output of two or more microfluidic pumps, or equivalent mechanisms for moving formulation, is combined into a homogeneous, substantially homogeneous, or partly homogeneous formulation. The mixing system comprises, in one embodiment, a mixing chamber, wherein the one or more formulations is mixed within the chamber. The mixing chamber is an enclosed cavity with entry points for the two microfluidic pumps to supply fluid, and with one exit point to supply the mixed formulation to the aerosolizer, wherein that exit point may be connected to an element including but not limited to, a third microfluidic pump, a wicking element, preferably a foam material, or a combination of a microfluidic pump and a wicking element. The mixing chamber may, in one embodiment, contain a mixing element, wherein this mixing element may be, but is not limited to, a piezoelectric actuator that causes disturbances in the liquid to encourage mixing. In a preferred embodiment this mixing chamber is the formulation chamber within the aerosolization head, wherein the mixing chamber is in direct fluid contact with the aerosolizer.

In another preferred embodiment, the mixing system is comprised of a multiplicity of mixing chambers, preferably 2 mixing chambers. An example of how this might work is as follows: A first formulation is introduced into a first mixing chamber, which already contains previously mixed formulation. This introduction forces some of the already mixed formulation into a second mixing chamber, and forces some of the mixed formulation in the second mixing chamber out of the mixing system and toward the aerosolization mechanism. Subsequently, a second formulation is then introduced into the second chamber, and some of the previously mixed formulation is forced out of the second chamber as before. Preferably the volume and shape of the first and second mixing chambers, and the passageways for the first and second formulations to enter, and the passageways for the mixed formulation to exit, are configured such that none of the unmixed first and second formulation exits the mixing system during this introduction phase. Subsequently, the inlet ports and the exit port from the second mixing chamber are closed off, and liquid is forced back and forth between the two chambers until the contents are fully mixed. This forcing can be accomplished with one or more pumps between the two mixing chambers. Preferably, the two mixing chambers themselves have the pumping functionality. For example, they may each have a flexible wall which can be forced inwardly into the chamber by an actuation mechanism. To pump back and forth, the entrances to the chambers and the exit from the second chamber are closed off, and alternately one and then the other actuation mechanisms are activated to cause pumping back and forth between the two chambers

In another embodiment, there is no mixing chamber, and the one or more formulations are mixed within the pumping system itself, preferably within the tubing, channels, or other passageways that comprise the pumping system, wherein the passageways may be characterized by, but not limited to, characteristics such as a long, relatively narrow passage, a tortuous path, vanes, paddles and the like (both moving and non-moving) in order to facilitate mixing. In a preferred embodiment, the one or more formulations that are pumped out of the one or more formulation chambers are pumped into one tube, channel, or other passageway, wherein the formulations mix within this passageway, whereupon the mixed or substantially mixed formulation is then delivered into the formulation chamber of the aerosolization head.

In a related embodiment, the pumps that deliver the two formulations from the formulation chamber(s) are configured as chambered micropumps, each with n chambers, where 1/n is the desired reformulation of dosing control. To titrate the dose, channels are opened which connect the distal most m chambers of the first formulation and the distal most n−m chambers of the second formulation, and the micropump are used to pump the formulation back and forth between the two lines, effecting mixing. By way of example, where n=10, m=0, 1, 2, 3, 4, 5, 6, 7, 8, 9, and 10 leads to formulation concentrations which are 100%, 90%, 80%, 70%, 60%, 50%, 40%, 30%, 20%, 10%, and 0% of the original active ingredient containing formulation concentration.

In one embodiment, the pumping system comprises two separate microfluidic pumps, wherein one microfluidic pump moves an active ingredient, preferably nicotine, containing first formulation, and one microfluidic pump moves a second, preferably non-active ingredient containing, formulation. The output from the microfluidic pumps is delivered directly to the formulation pocket of the aerosolization head, wherein this chamber may be an empty chamber or may comprise a wicking element. One embodiment may comprise an absorbent wicking element, wherein the output of each microfluidic pump, or equivalent mechanism for moving fluid, is provided to the wicking element. In another embodiment, the two formulations essentially completely mix in the formulation pocket of the aerosolization head and the aerosol formed by the aerosolizer is made up of droplets containing essentially same concentration of the at least one active ingredient. In another embodiment, no mixing, or alternatively incomplete mixing, occurs in the formulation pocket and the aerosol formed has particles with differing concentrations of the at least one active ingredient.

In another embodiment, the pumping system contains one microfluidic pump, wherein this pump is used to pump at least one formulation, preferably two or more formulations from separate formulation chambers, and a valve for controlling the flow of formulations from the one or more formulations chambers. In a preferred embodiment, one or more of passageways located upstream from the pump connect the one or more formulation chambers to a central passageway, preferably a tube, whereupon the central passageway then connects directly to the input of the microfluidic pump, proceeds to the outlet of the microfluidic pump, and is delivered to the mixing element, which is, preferably, the formulation pocket of the aerosolization head. In one embodiment, a valve is located at the convergence point where the one or more passageways conjoins into the central passageway upstream of the pump, wherein the position of the valve can be controlled via an electronic signal, wherein the valve is positioned to block the flow from all but one of the one or more formulation chambers, thus controlling which of the one or more formulations is pumped into the pumping system. In another embodiment, each of the one or more passageways that connect to the one or more formulation chambers contains an electronically controlled valve, wherein this valve may be positioned to the open position, wherein formulation is able to flow through the pumping system, or the closed position, wherein no formulation is able to flow through the pumping system. In another embodiment, the one or more passageways that connect to the one or more formulation chambers each contains a valve, wherein this valve can be positioned to partially restrict the flow of formulation through the passageway. In this embodiment, the position of the one or more valves is independently controlled to determine the flow of each of the one or more formulations to achieve a proper mixture of the formulations. In the above stated embodiments, the position of the valve(s) is controlled so as the achieve a particular mixture of the one or more formulations, wherein this mixture may be determined by one or more of the factors including, but not limited to, length of time, duration of use of the system, number of inhalations, and number of dosing events.

In a preferred embodiment, the system only contains a single formulation, and a single pump.

In one embodiment where microfluidic pumps in the durable system are used, there are at least two fluid connection points between the cartridge and the durable system, one for each formulation chamber, to allow for the formulation to flow from the formulation chamber(s) to the pump(s) in the durable system, and one to allow flow of formulation from the pumps to the aerosolization head when the cartridge is connected to the durable system. When a cartridge no longer contains a sufficient amount of formulation to enable proper use (henceforth referred to as a “spent cartridge”) is detached from the durable system of the device and replaced with a new cartridge, it is important that contaminants not be introduced into the system. It is also important to avoid air ingress, as this may interrupt proper aerosolization and require re-priming of the pumps. The fluid connection points may be a film of material, including, but not limited to, a metal foil, a polymer, or a mesh material, that is penetrated upon connection to the durable system. Preferred materials for the film include but are not limited to PTFE, polypropylene, and/or silicone. In another embodiment, the connection point may be a valve that is opened upon connection of the cartridge to the durable system. In a preferred embodiment, the connection point(s) may comprise at least one pierceable silicon septum, preferably two pierceable septa, wherein one is located on the surface of the durable system, referred to as the first septum, and one is located on a mating surface of the cartridge, referred to as the second septum, and a needle, preferably a non-coring needle, attached to one of the cartridges or the durable system. Upon attaching the cartridge to the durable system, the needle pierces the septa to create a water- and air-tight seal. Preferably, the needle may be protracted through and retracted from the septa upon connection and disengagement of the cartridge and durable system, preferably by the means of, but not limited to, a spring. In the embodiment where the needle is attached to the durable system, protraction and retraction of the needle through the septa may be achieved by 1) rigidly connecting the second septum to the cartridge, 2) slidably connecting the first septum to the durable system in a way that the spring is compressed and presses the first septum against a stop. Upon connection of the cartridge to the durable system, the first septum is translated in a way that compresses the spring and causes the needle to pierce the first septum, whereupon the needle protracts out from the first septum, pierces the second septum, and protracts out the other side of the second septum and into a channel or tube leading to a formulation chamber. In a preferred embodiment, at least one of the pierceable silicone septa, preferably the first septum, covers the tip of the needle when the cartridge is detached from the durable system, wherein this septum is self-healing and thus creates an airtight seal over the tip of the needle to prevent the ingress of such air and contaminants. This connection point may further be any other embodiment that, upon connection of the cartridge to the durable system, allows liquid to between the cartridge and the durable system, but does not allow liquid flow, evaporation, air ingress, or contamination of the formulation when the cartridge is not connected.

In one embodiment, the pump is a peristaltic pump comprised of components selected from but not limited to a motor, a gear head, a rotor or eccentric cam, an alignment cam, a ring, a section of flexible tubing, and a support surface for the tubing. In one embodiment of the peristaltic pump a motor turns a rotor, which rotor circularly translates at least one roller, whereupon the roller rolls along a piece of flexible tubing and compresses it against a supporting surface in order to create flow through the tubing. In another embodiment of the peristaltic pump, the motor turns an eccentric cam, wherein this eccentric cam is surrounded by a ring, characterized by being of sufficient diameter that the eccentric cam fits completely within the ring, and whereupon activation of the motor the eccentric cam rotates the eccentric cam and thereby translates the ring, causing the ring to sequentially compress sections of the tubing against a supporting surface in a circular pattern, thereby creating flow through the tubing. In an embodiment wherein the system is comprised of a durable system device and a separable multidose cartridge, it is preferable that the motor, gear head, and eccentric cam or rotor are part of the durable system and the tubing, ring, and support surface are part of the disposable.

In one embodiment, the pumping system contains one pump, preferably a peristaltic pump, wherein this pump is used to pump at least one formulation, preferably two or more formulations from separate formulation chambers, and a valve for controlling the flow of formulation from the formulation chambers. In one embodiment one or more passageways located upstream from the pump connect the one or more formulation chambers to a central passageway, preferably a tube, whereupon the central passageway then connects directly to the input of the peristaltic pump, proceeds to the outlet of the peristaltic pump, and is delivered to the formulation pocket. In one embodiment a valve is located at or near the convergence point where the one or more passageways conjoin into the central passageway upstream of the pump, wherein the state (flow allowed, flow blocked, optionally flow rate controlled) of the valve can be controlled mechanically and/or via an electronic signal, wherein the valve is positioned to block the flow from all, all but one, all but a subset, or none of the one or more formulation chambers, thus controlling which of the one or more formulations is pumped into the pumping system. In another embodiment each of the passageways of the one or more passageways that connect to the one or more formulation chambers contains an electronically controlled valve, wherein this valve may be positioned to the open position, wherein formulation is able to flow through the pumping system, or the closed position, wherein no formulation is able to flow through the pumping system. In another embodiment the one or more passageways that connect to the one or more formulation chambers each contains a valve, wherein this valve can be positioned to partially restrict the flow of formulation through the passageway. In this embodiment the position of each of the one or more valves is independently controlled to determine the flow of each of the one or more formulations to achieve a proper mixture of the formulations. In the above stated embodiments, the state of the valve(s) is controlled so as the achieve a particular mixture of the one or more formulations, wherein this mixture may be determined by one or more of the factors including, but not limited to, length of time, duration of use of the system, number of inhalations, and number of dosing events.

Preferably, the exit of the one or more formulation chambers are closed before the cartridge is first used to ensure that the one or more contained formulations are only in contact with the formulation chamber during storage and transport, and only in contact with any tubing, channels, pockets, or aerosolizers during and after first use. This allows the chamber materials to be selected for their long term storage properties, including but not limited to water (or other carrier) vapor transmission rate, leachables, extractables, and/or drug stability, and the materials of the other portions of the flow path can be chosen for other properties, including but not limited to mechanical flexibility, ease of manufacture, aerosolization performance, and the like.

Preferably, in the above-mentioned embodiment, elements selected from but not limited to a motor, gear head, electronics, battery, button, light, display, aerosolizer, eccentric, ring, or rotor of a peristaltic pump are components of the durable system, wherein elements selected from but not limited to an aerosolizer, a pump head comprised of elements selected from but not limited to a support surface, flexible tubing, rotor, eccentric, and a ring; one or more formulations, formulation chambers, passageways, tubes, filters, and debubblers, are all components of the cartridge. This ensures that the one or more formulations are never in direct fluid contact with any durable system component of the device and the drug contact surfaces are contained wholly in the cartridge. To accomplish this, the peristaltic pump motor will be located in the durable system such that, when the cartridge is attached to the durable system, tubing will be positioned to be compressed by with the outer diameter of the ring, both of which are parts of the cartridge, wherein upon actuation of the pump motor which is part of the durable system, the motor will turn the rotor or the eccentric cam of the pump, to sequentially compress sections of the tubing in order to create flow. This connection of the two important components of the pumping system, the peristaltic pump and the tubing, will be accomplished solely by attaching the cartridge to the durable system of the device.

The pumping system incorporates a supply mechanism for transporting the formulation, preferably a mixture of formulations, to the aerosolizer. This supply mechanism may incorporate micro-pumps, peristaltic pumps, or other active pumping components to pump the liquid from a mixing element to the aerosolizer. Preferably, the pumping mechanisms described previously are also used to supply the formulation to the aerosolizer, and the supply mechanism is essentially only one or more passageways. The pumping mechanism may supply formulation to the aerosolizer during aerosolization and at a flow rate which is substantially the same as the rate at which the aerosolizer consumes formulation, wherein, in one embodiment, this is accomplished by a flow channel that delivers formulation to a wicking element, wherein the formulation may be delivered to the middle, to the edge, or any other portion of the wicking element, and in another embodiment, this is accomplished by a flow channel that delivers formulation into the formulation pocket with no wicking element. The pumping mechanism may also supply formulation to the aerosolizer during, before, and after aerosolization at a flow rate which is greater than, equal to, or less than the aerosolization rate. Preferably the formulation pocket and other formulation containing volumes are free from any air-filled headspace. In this embodiment the formulation may be drawn from the formulation chamber during aerosolization and subsequently replenished as needed to prevent the creation of an air-filled headspace behind the aerosolizer.

The durable system contains elements selected from but not limited to a power source, which may be, but is not limited to, a battery or cell, preferably a rechargeable battery or cell which is preferably comprised of lithium; a printed circuit board, a charging port which is preferably mechanically attached to the printed circuit board and is accessible from the exterior of the device, a circuit for driving the liquid pumping system, a circuit for driving the aerosolizer, a light, a display, a button, and at least one pressure sensor. These circuit may be discrete subsystems on the circuit board, or may be incorporated in an application specific integrated circuit. Preferably the circuit board powers the functionality of the device and controls the aerosolizer and pumping system, and, in one embodiment, is comprised of a computing system that determines and controls the proper flow rate and dispensed volume for each microfluidic pump of the pumping system. The computing system controls an element selected from a list including but not limited to the flow rate of one or more pumps, the state of one or more valves controlling flow from one or more formulation chambers, the voltage supplied to an aerosolizer, the amplitude of oscillation of a component of an aerosolizer, a duration of pumping of one or more pumps, a duration of aerosolization of an aerosolizer, such that the amount of nicotine, or other active ingredient, delivered to the user is consistently lowered over time. For example, upon the initial use of the device, the device may be controlled such that an initial total emitted dose of nicotine per average inhalation is in a range selected from a list including but not limited to from about 0.05 to about 2 mg, from about 0.1 to about 1 mg, from about 0.2 to about 0.5 mg, from about 0.25 to about 0.3 mg, whereupon the amount of nicotine will be decreased, for example the flow rate of a nicotine containing formulation will be decreased and the flow rate of a non-nicotine containing formulation increased, such that after a period of time selected from about 1 week to about 1 year, about 1 month to about 6 months, about 2 months to about 4 months, preferably about 3 months, the total emitted dose of nicotine per average inhalation may be approximately 0 mg, and for example wherein the total aerosolized volume of formulation is substantially consistent. This serves as an example only for the purpose of clarification. Preferred starting amounts of nicotine per puff are preferably greater than or about 2 mg/puff, greater than or about 1.5 mg/puff, greater than or about 1.2 mg/puff, greater than or about 1 mg/puff, greater than or about 0.8 mg/puff, greater than or about 0.6 mg/puff, greater than or about 0.5 mg/puff, greater than or about 0.4 mg/puff, or greater than or about 0.3 mg/puff. Ending amounts of nicotine are preferably less than or about 1 mg/puff, less than or about 0.8 mg/puff, less than or about 0.7 mg/puff, less than or about 0.6 mg/puff, less than or about 0.5 mg/puff, less than or about 0.4 mg/puff, less than or about 0.3 mg/puff, less than or about 0.2 mg/puff, less than or about 0.1 mg/puff, or about 0 mg/puff.

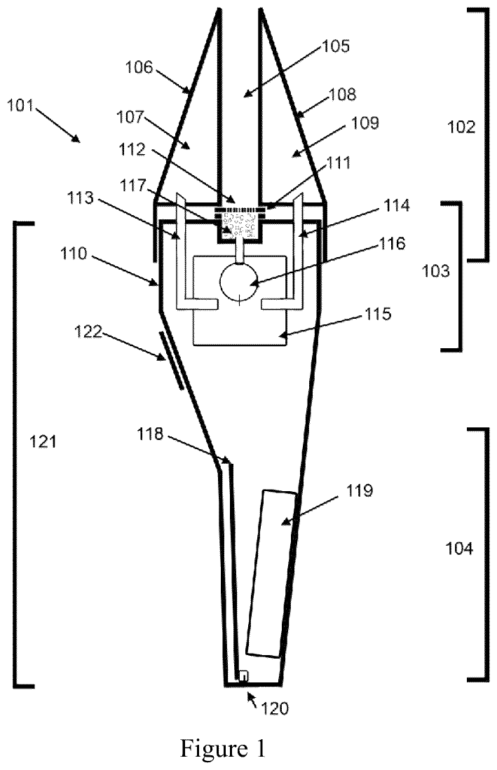

As detailed below, the components of the aerosol device are detailed in the accompanying figures. FIG. 1 shows an embodiment of aerosol device 101 of the current invention. Aerosol device 101 comprises mouthpiece section 102, microfluidics and aerosol section 103, and electronics section 104. Mouthpiece section 102 comprises inhalation channel 105, first formulation container 106 containing first formulation 107, and second formulation container 108 containing second formulation 109. Housing 110 contains microfluidics and aerosol section 103 and electronics section 104. Mouthpiece 102 is attached to housing 110 in such a way that inhalation channel 105 is aligned with nozzle array 112 which is formed in substrate 111.

First feed channel 113 connects to first formulation container 106 and first formulation 107 is delivered to microfluidics board 115, and second formulation 109 is delivered to microfluidics board 115 in a similar manner. First formulation 107 and second formulation 109 are mixed in mixing chamber 116 then delivered to nozzle array 112 via wicking element 117.

The microfluidics components on microfluidics board 115 are controlled by electronics on printed circuit board assembly (PCBA) 118, which is powered by battery 119. Battery 119 is rechargeable and recharged by connecting a charging cable (not shown) to charging receptacle 120.

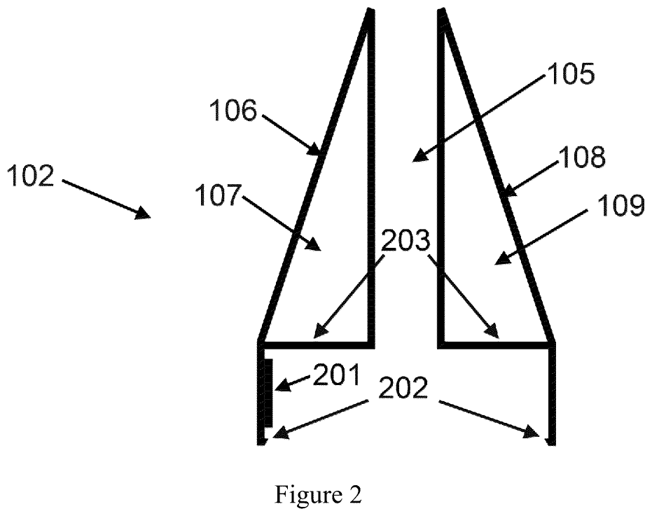

FIG. 2 is a more detailed view of mouthpiece 102. In this embodiment, mouthpiece 102 is comprised of key 201. When mouthpiece 102 is attached to housing 110, key 201 ensures that mouthpiece 102 can only be attached in the correct orientation. Mouthpiece 102 is also comprised of latching features 202 which ensure that mouthpiece 102 is positively but removably attached to housing 110. Preferred latches 202 provide feedback to the user that mouthpiece 102 is correctly attached to housing 110. Preferred feedback includes, but is not limited to, an audible click and/or a tactile click. Item 203 is a pierceable element, preferably a membrane. When mouthpiece 102 is attached to durable section 121, feed tubes 113 and 114 pierce pierceable membrane 203, enabling flow of formulation to microfluidics board 115.

First formulation container 106 contains first formulation 107. In one embodiment, first formulation 107 is in direct contact with the walls of first container 106, and the walls of first container 106 have the required container closure properties comprising acceptable extractables, acceptable leachables, materials that are consistent with the stability for first formulation 107, acceptably low binding of key components of formulation 107, and are essentially impermeable to the carrier of formulation 107. In one embodiment, the carrier comprises water, and/or ethanol. Second formulation container 108 has essentially the same properties with respect to second formulation 109.

In one embodiment, formulation 107 is not in direct contact the walls of container 106, but rather the walls of container 106 enclose and protect a primary container closure system for first formulation 107. The container closure system has the required properties comprising acceptable extractables, acceptable leachables, materials that are consistent with the stability for first formulation 107, acceptably low binding of key components of formulation 107, and is essentially impermeable to the carrier of formulation 107. In one embodiment, the carrier comprises water and/or ethanol.

The container closures 106 and 108 may be essentially rigid and comprise a material including, but not limited to, glass, preferably borosilicate glass, a metal, preferably stainless steel, or a polymer. The container closure system may comprise of at least one wall which is flexible and is comprised of a polymer. A flexible container has the advantage that it does not create back pressure on the pump or require air ingress into formulation container 106 and ensures that formulation can be pumped out of formulation container 106 in a way that is independent of orientation and gravity. In a preferred embodiment, the at least one flexible wall is a multi-layer laminate and may be comprised of one or more layers including but not limited to a drug contact layer, preferably comprised of a relatively inert polymer which may be but is not limited to polyethylene, or a fluoropolymer, preferably polytetrafluoroethylene (PTFE); a vapor transmission layer preferably comprised of a metal, preferably aluminum; and a structural layer, preferably a polyamide such as a Nylon. Second formulation container 108 preferably has a container closure system with essentially the same properties with respect to second formulation 109.

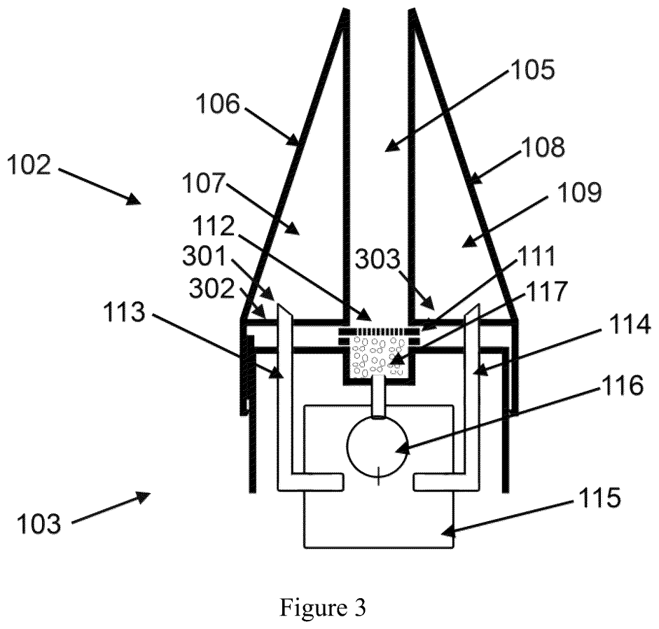

FIG. 3 shows a detail of mouthpiece 102 attached to microfluidics and aerosol section 103 of an embodiment of the current invention. First feed channel 113 is in fluidic contact with first formulation 107 in formulation container 106 so that it may draw out a portion of first formulation 107 as needed. In the embodiment shown in FIG. 3, sharpened end 301 extends through first pierceable septum 302. Preferably sharpened end 301 is non-coring. Other ways of creating a fluid connection to formulation 107 may be used, for example a hole or connector into which first feed channel 113 is fed. In one embodiment, mouthpiece 102 is supplied with a cap that prevents leakage from and contaminant ingress into a hole or connector in septum 302. Preferably, the hole or connector is such that the hole or connector is shut or sealed prior to the insertion of first feed channel 113 into formulation container 106. A similar discussion applies to second formulation container 108, second formulation 109, second flow channel 114, and second septum 303.

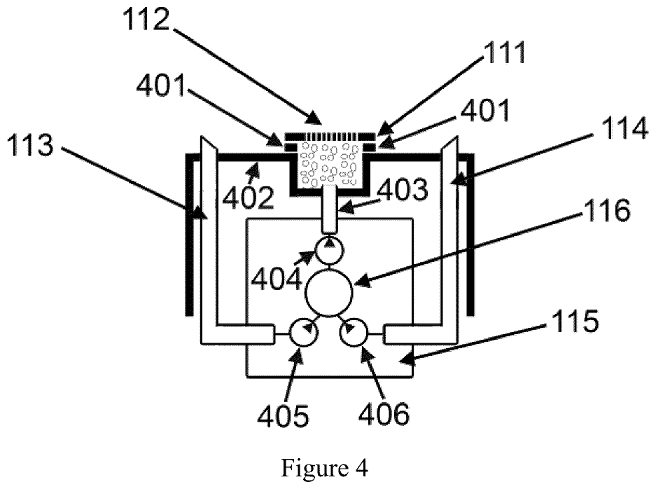

FIG. 4 shows the microfluidics and aerosol section 103 in more detail. Formulation 107 is drawn through feed channel 113 by micropump 405, and pumped into mixer 116. In one embodiment, first micropump 405 is a piston type micropump. First micropump 405, under the control of a controller on PCBA 118, cycles m times, dispensing a volume of first formulation 107 equal to m*V1, where V1 is the stroke volume of first micropump 405, into mixing chamber 116. Similarly, second micropump 406 dispenses a volume (n−m)*V2 of second formulation 109 into mixer 116, where V2 is the stroke volume of second micropump 406. Preferably V1=V2=V. In one embodiment, first pump 405 pumps m cycles, and subsequently second pump 406 pumps n−m cycles. In another embodiment, second pump 406 pumps n−m cycles, and subsequently first pump 405 pumps m cycles. In a preferred pump embodiment, pumps 405 and 406 pump essentially simultaneously into mixer 116 to optimize mixing. After mixing, pump 404 delivers the mixed formulation via tube 403 to the aerosolizer. In the embodiment shown, the aerosolizer is a vibrating mesh aerosolizer comprised of piezo 401, substrate 111, and array of holes 112. Piezo 401 is mounted to housing 402.

In one embodiment, n is essentially fixed and unchanging over the lifetime of device 101, and m is varied to vary the amount of one or more active ingredients in the delivered aerosol. In another embodiment, n and m are variable, and the pumping is activated after each dosing event. n and m will in general vary between different dosing events, due to one or more factors that may include but are not limited to a desired delivered dose of one or more active ingredients, a desired delivered concentration of one or more active ingredients; a number of puffs taken in one or more previous dosing events, preferably the number of puffs taken in the previous dosing event; the total combined duration of the puffs taken in one or more previous dosing events, preferably the total combined duration of the puffs taken in the previous dosing event; the expected the number of puffs to be taken in one or more future dosing events, preferably the expected number of puffs to be taken in the next dosing event; the expected total combined duration of the puffs expected to be taken in one or more future dosing events, preferably the expected total combined duration of the puffs to be taken in the next dosing event; the current aerosolization rate from nozzle array 112; and/or the level of saturation of wick 117.

In a preferred embodiment, n and m are variable, and pumping is initiated after each puff. The values of n and m will in general vary between different puffs, due to factors that may include but are not limited to a desired delivered dose of one or more active ingredients, a desired delivered concentration of one or more active ingredients; the number of puffs taken in one or more previous dosing events, preferably the number of puffs taken in the previous dosing event; the total combined duration of the puffs taken in one or more previous dosing events, preferably the total combined duration of the puffs taken in the previous dosing event; the expected number of puffs to be taken in one or more future dosing events, preferably the expected number of puffs to be taken in the next dosing event; the expected total combined duration of the puffs expected to be taken in one or more future dosing events, preferably the expected total combined duration of the puffs to be taken in the next dosing event; the current aerosolization rate from nozzle array 112; the level of saturation of wick 117; the duration of the previous puff; and/or the expected duration of the next puff.

In a particularly preferred embodiment, V1=V2=V, and after each puff, first formulation 107 is pumped by first pump 405 in an amount equal to m*V, second formulation is pumped in an amount equal to (n−m)*V, and n*V is equal to the volume of mixed formulation removed from wick 117 during the previous puff.

Mixing chamber 116 is shown as an essentially round chamber. However mixing chamber 116 can be of any shape, including but not limited to round, oval, rectangular, disc shaped, spherical, hemispherical, or combinations or sections thereof. Preferred shapes are those that facilitate mixing, including but not limited to long, thin channels and or sinuous shapes. First formulation 107 and second formulation 109 may be introduced into mixing chamber in essentially any way, but preferably are introduced in a way that optimizes mixing of formulations 107 and 109. The introduction into mixing chamber 116 may range from essentially toward the center of mixing chamber 116 to essentially tangentially to a wall of mixing chamber 116. In a preferred embodiment, mixing is optimized by introducing formulation into mixing chamber 116 in a way that the directions of flow of first formulation 107 and second formulation 109 intersect in mixing chamber 116 and have a substantial component toward each other, preferably the lines of flow (i.e. the vectors which are the center lines of the flow of first formulation 107 and second formulation 109) as they enter mixing chamber 116 intersect at an angle greater than or about 45°, preferably at an angle greater than or about 60°, at an angle greater than or about 90°, at an angle greater than or about 120°, or at an angle greater than or about 160°. In a preferred embodiment the lines of flow of first formulation 107 and second formulation 109 as they enter mixing chamber 116 are directed substantially directly at each other.

It will be noted that although the fluidics system in this and subsequent embodiments may be shown as a board and referred to as a fluidics board or a micro-fluidics board, it will be understood that there can be different embodiments for the fluidics system, including but not limited to: embodiments that do not use micro-fluidics, embodiments that include more than one board, and/or embodiments that are comprised of discrete components that are not mounted on a board.

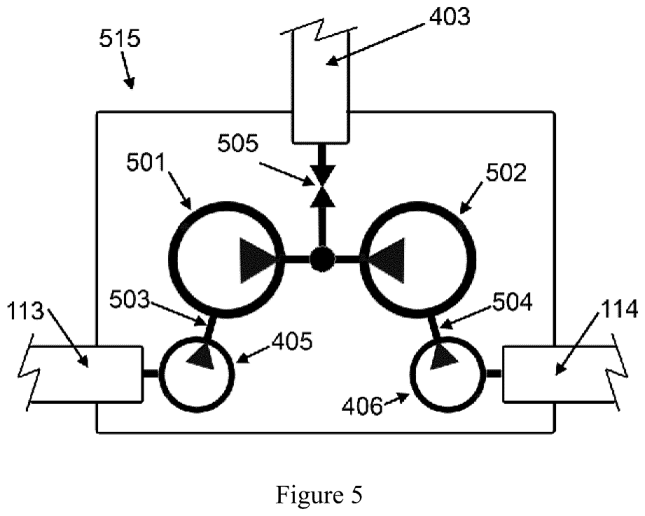

FIG. 5 shows another embodiment comprising fluidics board 515. Fluidics board 515 is comprised of first large pump 501 and second large pump 502. In one embodiment, the stroke volume of each of large pumps 501 and 502 is preferably greater than or about equal to m*V1+(n−m)*V2. In another embodiment, the stroke volume plus the dead volume of each of large pumps 501 and 502 is preferably greater than or about equal to m*V1+ (n−m)*V2. The functioning of the fluidics board 515 is as follows: First formulation 107 is pumped utilizing m cycles of first pump 405 through first channel 503 into first large pump 501. Second formulation 109 is pumped utilizing n−m cycles of second pump 406 through second channel 504 into second large pump 502. Valve 505 is closed, and then pumps 501 and 502 are sequentially actuated to move the formulation from large pump 501 to large pump 502, and then back to pump 501. This cycle is repeated until the formulation is sufficiently mixed. Finally valve 505 is opened, and large pumps 501 and 502 are simultaneously actuated, forcing the mixed formulation into wick feed channel 403.

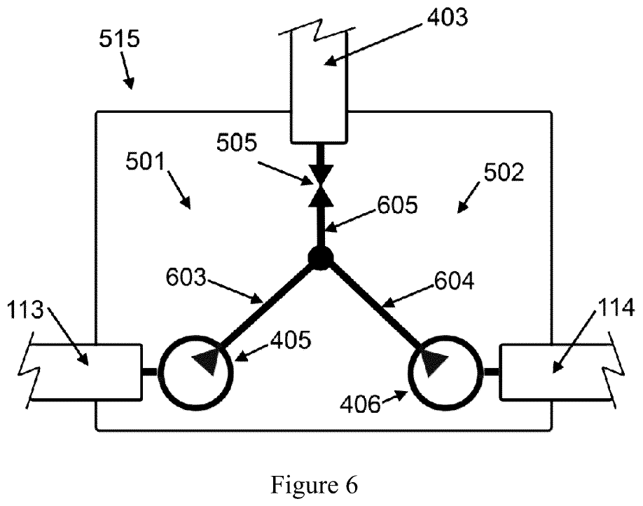

FIG. 6 shows an embodiment of the current invention that does not have a mixing chamber. This embodiment might be used for reasons including but not limited to sufficient mixing occurs in channel 605, sufficient mixing occurs in channel 403, sufficient mixing occurs in wick 117, and/or mixing is not required. Mixing might not be required, for example, if sufficient control of the amount of one or more active ingredients can be achieved, wherein it is acceptable for the amount and/or concentration of the one or more active ingredients in the aerosol particles to vary across the aerosol plume. The operation of the embodiment of FIG. 6 is similar to the above embodiments. Optional valve 505 is opened, and then m cycles of first pump 405 pump an amount m*V1 of first formulation 107 through channel 603. Subsequently, or preferably concurrently, n−m cycles of second pump 406 pump an amount n*V2 of second formulation 109 through channel 604. Finally, optional valve 505 is closed.

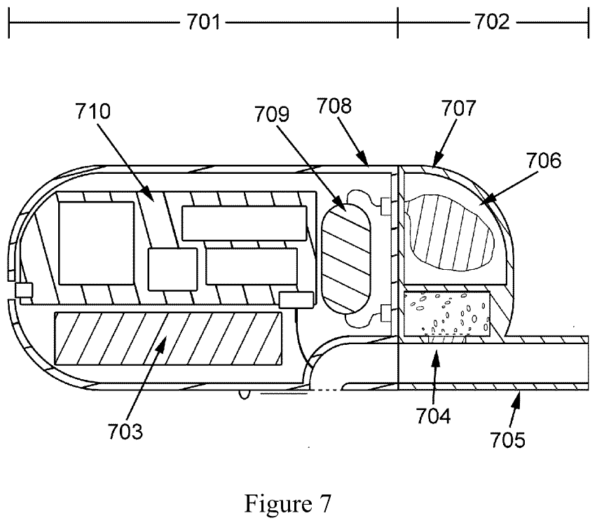

FIG. 7 shows another embodiment of the current invention comprised of durable system section 701 and multidose cartridge 702 of the current invention. In the embodiment shown, durable system section 701 is comprised of electronics board 710, one or preferably two or more microfluidic pumps 709, and battery 703 contained within housing 708. Cartridge 702 is comprised of at least one formulation cavity 706, aerosolization head 704, and mouthpiece 705, contained within housing 707.

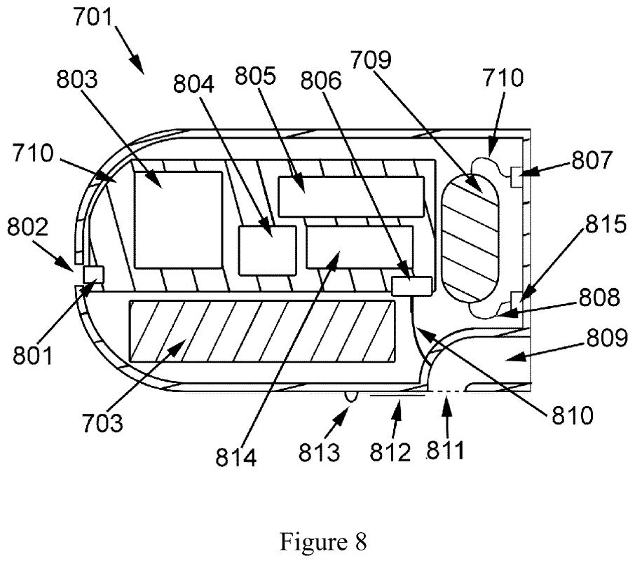

FIG. 8 is a more detailed view of durable system section 701. In the embodiment shown, electronics board 710 is comprised of microcontroller 803, which controls battery charging circuit 804, pump controller 805, and mesh piezo controller 814, and measures airway pressure using pressure transducer 806. It will be understood that electronics board 710 may actually be comprised of two or more separate boards and/or discrete components that are not on an electronics board. Programming of microcontroller 803 is achieved through connector 801, preferably USB connector 801, accessed by a cable through housing opening 802. Preferably, the cable also supplies the current to charge battery 703. Pump controller 805 controls at least one microfluidic pump 709. Pumps 709 receive formulation from formulation cavities 706 via liquid connection 807 and tubing 710. Liquid formulation is delivered to aerosolization head 704 by pump 709 via tubing 808 and liquid connection 815. Housing 708 is comprised of the entry section to the inhalation airway 809. The inlet to airway 809 is covered by mesh 811. Mesh 811 may be a series of machined or molded slots in housing 708, or may be a separate component attached to housing 809. Mesh 811 serves two purposes: One is to keep the airway clear of objects that are large enough to be dangerous if inhaled. Another purpose is to create a pressure drop that can be measured in order to only generate aerosol if the user is inhaling at an optimal flow rate relatively early in the inhalation. In the embodiment shown in FIG. 8, the pressure in airway 809 is measured by pressure transducer 806 via pressure tube 810. Based on the signal received from pressure transducer 806, and a previously conducted measurement of the airflow/pressure relationship of mesh 811, microcontroller 803 can determine if the user is inhaling at the optimal inhalation flow rate, and by integrating this flow rate over time the controller can determine the user's inhaled volume. If microcontroller 803 determines the user has achieved the correct flow rate early in the inspiration, the controller turns on the aerosol generation via mesh piezo controller 814, and keeps the aerosol generation on for a time elected from a list including but not limited to one or more of: a predetermined time that is constant during the therapy, a predetermined time that varies during the course of the therapy, for example to reduce the dose of nicotine over the course of the therapy, and a time which is shorter than the predetermined time for a reason selected from a list including but not limited to the user stops inhaling, the user begins to inhale at a lower than optimal flow rate, the user stops holding down a button.

During, or preferably subsequent to inhalation, microcontroller 803 can determine the amount of aerosol that has been generated and based on this the prescribed therapy and a dosing algorithm, microcontroller 803 can turn on pump 709 for a determined amount of time and/or pumping cycles. In a preferred embodiment microcontroller 804 turns on a second pump 709 and pumps a second formulation for a fixed, preferably different amount of time.

Durable system 701 also includes one or more buttons 812. Button 812 can be pushed to accomplish a task selected from the list including but not limited to: turning on the system, turning off the system, displaying the status of the system, informing the system that a replacement of cartridge 702 is complete, and the like. In a preferred embodiment, switch 812 is only used to turn the device on or “wake up” the device, electronics board 710 is comprised of wireless communication functionality, and all other user communication with controller 803 is done with a display device, for example a phone, computer, tablet, and the like. In another embodiment button 812 is pressed to wake up the device or ready the device to deliver a puff and the user must continue holding down the button. This can be useful if the button is situated in such a way that the device must held in a prescribe way selected from a list including but not limited to a prescribed orientation, a prescribed placement of the user's fingers and hand, for example to ensure that the user does not block air inlet 811. Durable system 701 is also comprised of light, preferably LED, 813. LED 813 may be one color, but preferably is multi-color and/or capable of flashing to communicate multiple different type of information selected from the list including but not limited to: The device is on, the inhalation flow rate is correct, the inhalation flow rate is incorrect (too low or too high), the battery requires charging, the battery charging is complete, the user should consult a companion app for additional information, and/or cartridge 702 needs to be replaced.

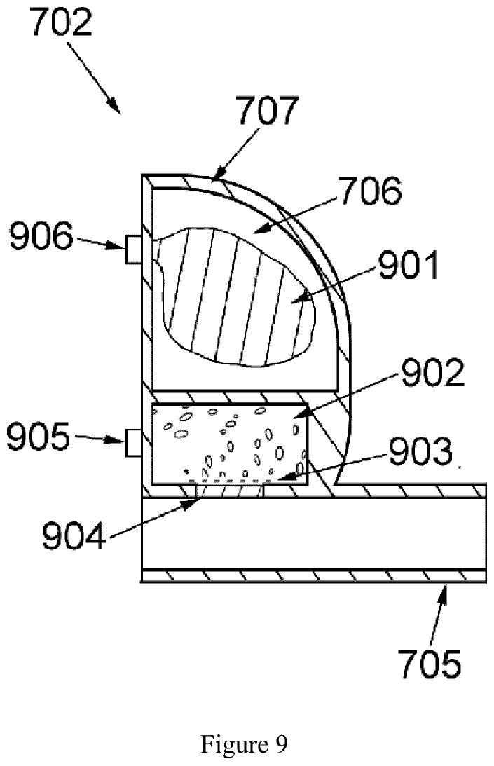

FIG. 9 shows an example of the multidose cartridge of the embodiment of FIG. 7. A formulation, preferably two or more formulations, are contained within formulation chambers 901 situated within formulation cavity 706. Preferably formulation chambers 901 are filled without a gas headspace, for example by vacuum filling, to ensure that the device usage is orientation independent and to ensure that gas from a headspace is not pulled into the system, resulting in low delivered dose and/or loss of prime. Preferably flexible formulation chambers 901 are flexible bags that are comprised of a pharmaceutically compatible drug contact surface, preferably polyethylene, a mechanical layer such as polyester or mylar, and a vapor transmission layer, preferably aluminum. The fluidic connection to the durable system unit is made via liquid connection 906.

The aerosolization head is comprised of piezo driven mesh 904 and optional wicking element 902. The use of wicking element 902 ensures that the formulation is delivered to mesh 904 independent of device orientation. Liquid formulation, preferably 2 or more liquid formulations, are delivered to mesh 904 via liquid connection 905.

It has been found that aerosol performance is optimized by having a small gap between wicking element 902 and mesh 904. The gap can be air, but preferably is created by including non-vibrating mesh 903. Non-vibrating mesh 903 can be a metal or plastic wire or otherwise porous mesh. In a preferred embodiment, mesh 903 is comprised of a very thin section of a very porous sponge material, for example more porous and/or containing larger pores than wicking element 902. Vibrating mesh 904 may be held at a small angle, for example about 5 degrees or less, about 2.5 degrees or less about 1 degree or less, or about 0.5 degree or less, relative mesh 903. In another embodiment, vibrating mesh 904 and mesh 903 are both held at a small angle, for example about 5 degrees or less, about 2.5 degrees or less, about 1 degree or less, or about 0.5 degree or less, relative wicking element 902.

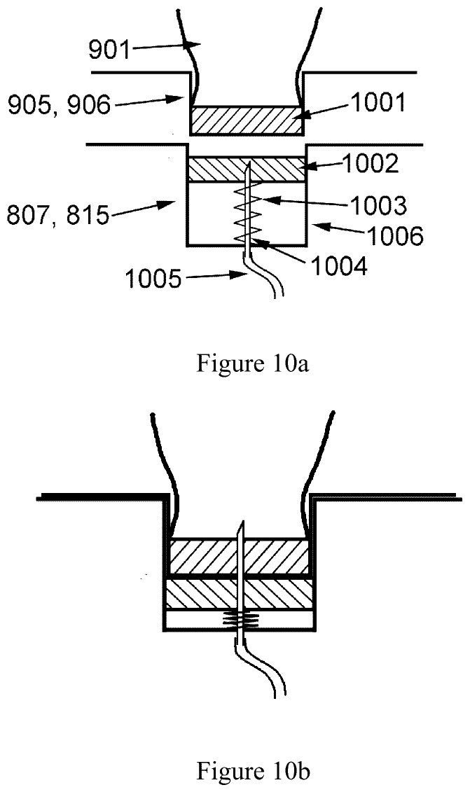

FIG. 10 shows an embodiment of the liquid connectors 905, 906, and mating connectors 815 and 807. It is important to minimize, preferably eliminate, the ingress of air and other contaminants into the fluidic system of durable system 701 and into formulation chambers 901. Air in the system can cause loss of prime and reduced dose. Contaminants can expose users to toxics, bacteria, or viruses. Dust or other particulates can block nozzle holes.

FIG. 10a shows an embodiment of the liquid connection in the state where durable system 701 and cartridge 702 are separated, either just before attachment or just after removal of the cartridge. Formulation chamber 901 (or wicking element 902 in the case of fluid connectors 815 and 906) is kept free from air and other contaminants by self-sealing pierceable septum 1001. The fluidics in tubing 1005 and other fluidic components of durable system 701 are similarly kept free of air and other contaminants by septum 1002. The tip of non-coring needle 1004 is sealed by being embedded in self-sealing pierceable septum 1002.

FIG. 10b shows the embodiment of FIG. 10a in the state where cartridge 702 is attached to durable system 701. When cartridge 702 and durable system 701 are pressed together, septum 1001 enters septum pocket 1006 and contacts septum 1002. Further pressing pushes septum 1002 further down into pocket 1006, compressing spring 1003. When durable system 101 and cartridge 102 are fully pressed together (preferably with a tactile and/or audible click to give feedback that cartridge 702 is properly attached to durable system 701, septa 1001 and 1002 are pushed into pocket 1006 far enough that the tip of needle 1004 is exposed, allowing microfluidic pump 709 to draw liquid from chamber 901 or pump liquid into wicking element 902.

When cartridge 702 is spent, an indication is sent to the user by light 813 and/or by a message (preferably accompanied by a tone and/or vibration) on the display device, preferably a smart phone. The user pulls cartridge 702 and durable system 701 apart, releasing septum 1002, which under the urging of spring 1003 returns septum 1002 to the position shown in FIG. 10a.

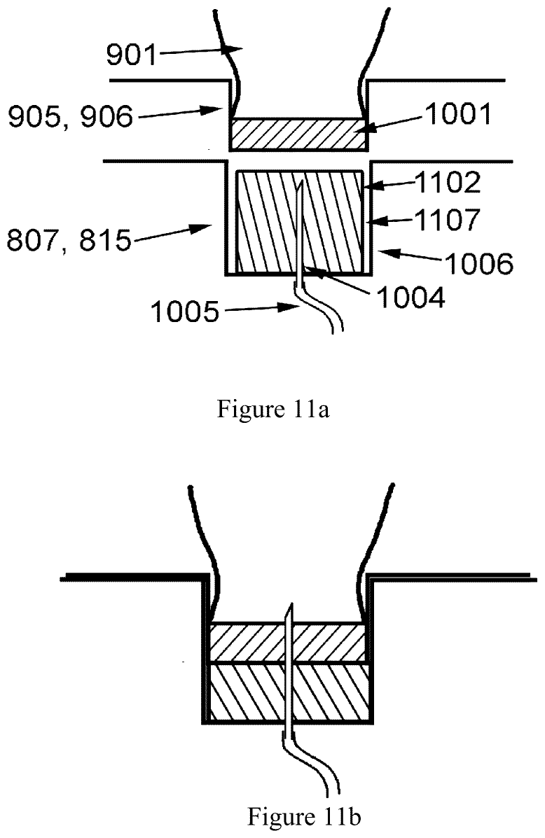

FIG. 11 shows an alternate embodiment of the fluid connector. In this embodiment, spring 1003 has been removed, and its functionality has been replaced by the compliance of longer septum 1102. FIG. 11a shows the state of the fluid connector when durable system 701 and cartridge 702 are separated, and FIG. 11b shows them connected. Gap 1107 is included to account for the expansion, due to its Poisson ratio, of septum 1102 when it is compressed. The compliance of septum 1102 can be tuned by changing the length of septum 1102 (with a concomitant change in the depth of pocket 1006), the width of septum 1102, the material of septum 1102, and the width of gap 1107.

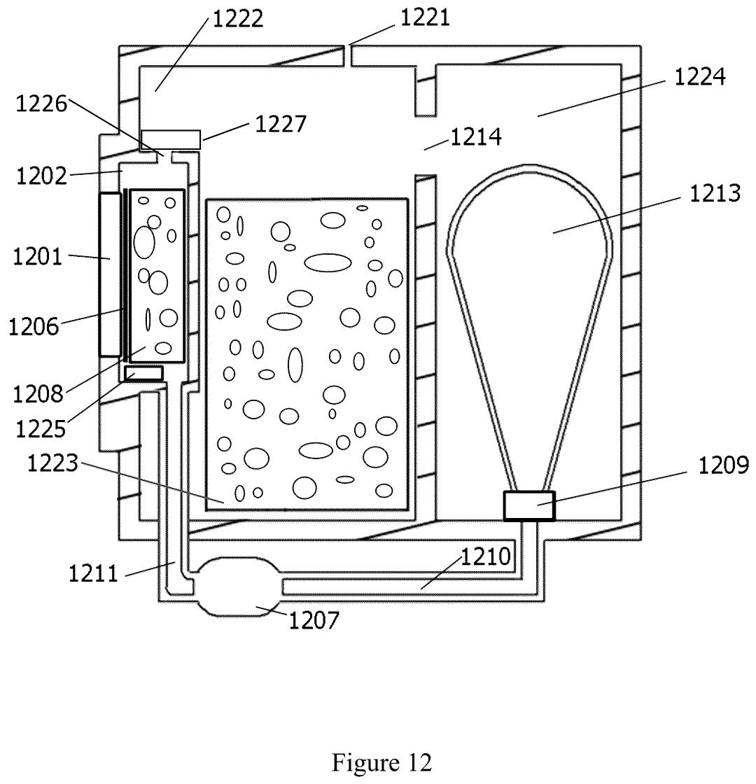

FIG. 12 shows an embodiment of the cartridge. One or more flexible formulation chambers 1213 are connected to one or more pumps 1207 via tubing 1210. Pump 1207 pumps formulation into tubing 1211, which carries formulation into formulation pocket 1202. During the process of aerosolization, aerosolizer 1201 generates air that goes into formulation pocket 1202. As pump 1207 pumps formulation into formulation pocket 1202, the air exits formulation pocket 1202 via opening 1226. Opening 1226 is covered by hydrophobic porous element 1227, which allows the air to escape, but as long as the pressure does not exceed a certain threshold, will not allow formulation to escape. Pressure transducer 1225 is supplied inside of formulation pocket 1202. Preferably pump 1207 pumps until the pressure as measured by pressure transducer 1225 is in a predetermined range, high enough to keep water in contact with aerosolizer 1201 and to expel water through hydrophobic porous element 1227, but not so high that formulation is expelled through hydrophobic porous element 1227 or aerosolizer 1201. Optionally, aerosolizer 1201 can be treated with a hydrophobic coating to further lessen the probability that the pressure in formulation pocket 1202 will force liquid out of the nozzle holes. Aerosolizer 1201 is preferably comprised of a nozzle hole, preferably a multiplicity of nozzle holes, preferably between about 100 and about 10,000 nozzle holes, or between about 500 and about 5,000 nozzle holes, or between about 1,000 and about 4,000 nozzle holes, or between about 1,000 and about 3,000 nozzle holes, preferably about 1,600 nozzle holes or 2,000 nozzle holes. Preferably, the nozzle holes are substantially round, with a diameter of less than or about 10 μm, or less than or about 7.5 μm, or less than or about 6 μm, or less than or about 5 μm, or less than or about 4 μm, or less than or about 3 μm, or less than or about 2 μm, preferably in the range of about 2 μm to about 3 μm. In one embodiment, the maximum pressure in formulation pocket 1202 is less than or about 100 mbar, less than or about 50 mbar, less than or about 25 mbar, less than or about 10 mbar, less than or about 5 mbar, less than or about 2.5 mbar.

During storage and prior to the first uses of the cartridge, valve 1209 is kept closed, ensuring the stability and sterility of the formulation in formulation chamber 1213. Valve 1209 may operate by pinching off tube 1210. In a preferred embodiment, valve 1209 operates by pinching off the exit formulation chamber 1213, so that the formulation is only in contact with formulation chamber during storage. Valve 1209 may be opened electrically under control of a microcontroller. In another embodiment, valve 1209 is opened by the user prior to the first use, for example, by a method selected from the list that includes, but is not limited to, pulling a pull tab, pressing a button, turning a knob, or the like. Preferably, the valve is opened by the act of attaching cartridge 702 to durable system 701. In the embodiment where valve 1209 is electrically actuated, it may be closed between uses to prevent the flow of formulation. Preferably, valve 1209 stays open for the entire time cartridge 702 is attached to durable system 701, to take advantage of the fact that flexible formulation chamber 1213 can maintain the system in pressure equilibrium with the surrounding air since small hole 1321 keeps the formulation cavity 1224, and if valve 1209 is open, all of the formulation in the cartridge at the same pressure as the surrounding air, minimizing the possibility of leakage, for example through aerosolizer 1201 or hydrophobic porous membrane 1227.

Before the first use of the current invention, valve 1209 is opened, and pump 1207 self-primes and then pumps the formulation though the recirculation system for sufficient time that essentially all of the air in formulation pocket, tubes 1210, 1211, 1315, and 1305, debubbler 1304, and pump 1207 is forced out of the cartridge, via aerosolizer 1201 and/or debubbler 1304. Pump 1207 self-primes and then pumps the formulation into formulation pocket 1202 until pressure transducer 1225 determines that the pressure in formulation pocket is at a predetermined pressure, insuring that essentially all of the air in formulation pocket 1222, tubes 1210, 1211, and pump 1207, is forced out of the cartridge, via aerosolizer 1201 and/or hydrophobic porous membrane 1227.

Small hole 1221 allows the pressure in formulation pocket 1222 and formulation cavity 1224 to equilibrate with the outside air, while being small enough maintain a high relative humidity in aerosol head cavity 1222 and formulation cavity 1224, thereby reducing evaporation of formulation carrier through hydrophobic porous membrane 1227. Because flexible formulation chamber 1213 is equilibrated with the outside air, the formulation in the device is maintained substantially at the same pressure as the outside air, substantially reducing or eliminating pressure differences across the nozzle holes of aerosolizer 1201, for example due to weather or changes in altitude, that can cause leakage of formulation or air ingress. In a related embodiment, hydrophobic porous membrane 1227 removes air from the formulation into a small cavity, which cavity vents directly to the outside air, or to aerosol head chamber 1222, or directly to formulation cavity 1224, preferably through a small hole as described above to maintain high relative humidity in the debubbler cavity and thereby reduce evaporation of formulation carrier through porous hydrophobic element 1227. In the event that formulation does exit the hydrophobic porous membrane 1227, optional absorbent material 1223 may be used to contain the leaked formulation. Preferably absorbent material 1223 is not required.

The walls of formulation pocket 1202 may contain a multiplicity of openings 1226 and hydrophobic porous membranes 1227, preferably on different faces of formulation pocket 1202, or the walls of formulation pocket 1202 may themselves be constructed of the material of hydrophobic porous membrane 1227, ensuring that air is cleared from pocket 1202 when the device is held in any orientation.

Optionally, one may choose to use wicking element 1208. In this embodiment, pump 1207 pumps formulation into tubing 1211, which carries formulation into formulation pocket 1202, wherein the formulation is absorbed by wicking material 1208, which is positioned within formulation pocket 1202 directly behind the aerosolizer 1201. The use of wicking element 1208 may make the device less orientation dependent. Optionally, wicking element 1208 may be separated from aerosolizer 1201 by spacing element 1206, which is positioned in direct contact with aerosolizer 1201. The use of spacing element 1206 is as follows: formulation flows from wicking element 1208, through the spacing element 1206, and to aerosolizer 1201. Upon actuation of aerosolizer 1201, aerosolizer 1201 pumps air into formulation pocket 1202. The air travels through spacing element 1206 into formulation pocket 1202, wherein the air exits formulation pocket 1202 via opening 1226 and hydrophobic porous membrane 1227.

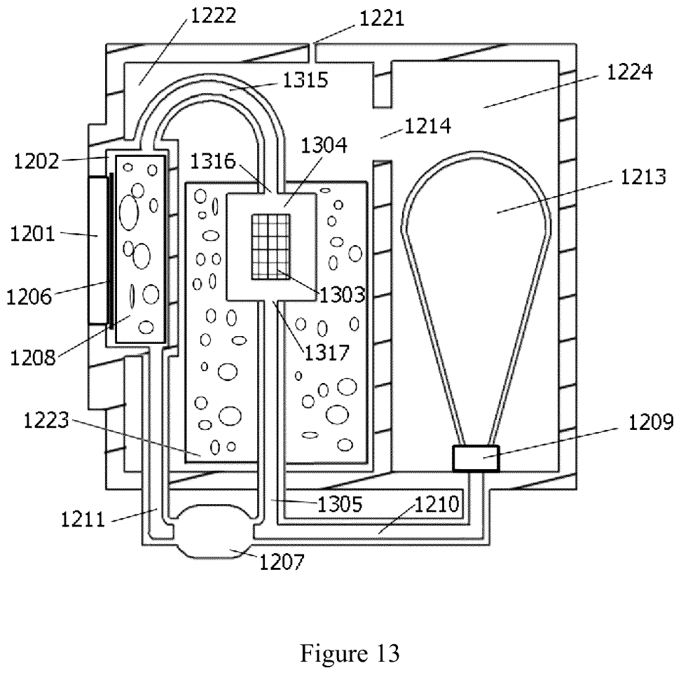

FIG. 13 shows an embodiment of the cartridge that uses a recirculation system. One or more flexible formulation chambers 1213 are connected to one or more pumps 1207 via tubing 1210. Pump 1207 pumps formulation into tubing 1211, which carries formulation into formulation pocket 1202. During the process of aerosolization aerosolizer 1201 generates air that goes into formulation pocket 1202. As pump 1207 pumps formulation into formulation pocket 1202, the air is entrained in the liquid flow and exits formulation pocket 1202 via tubing 1315, wherein formulation and air flow into debubbler 1304 via entrance opening 1316. The formulation passes through debubbler 1304 and out exit opening 1317. As the flow of formulation and air passes through debubbler 1304 it comes into direct contact with porous hydrophobic material 1303, wherein the air passes out of the porous hydrophobic material 1303 and then is pulled through opening 1214 into the reduced pressure (due to formulation being pumped out of formulation chamber(s) 1213) environment of formulation cavity 1224. Exit 1317 is preferably somewhat restrictive, such that the pressure in debubbler 1304 is sufficiently high to force the air through porous hydrophobic material 1303, but not so high that formulation passes through porous hydrophobic material 1303. The formulation then flows into tubing 1305 which connect to tubing 1210, wherein the formulation then is pumped back through pump 1207, thus creating a recirculating flow system. In general, the pressure in formulation pocket 1202 can be kept quite low, substantially equal to the pressure in debubbler 1304, reducing the possibility that formulation will flow through the array of nozzle holes in aerosolizer 1201. Optionally, aerosolizer 1201 can be treated with a hydrophobic coating to further lessening the probability that the pressure in formulation pocket 1202 will force liquid out of the nozzle holes. Preferably the pressure in formulation pocket 1202 is less than or about 100 mbar, less than or about 50 mbar, less than or about 25 mbar, less than or about 10 mbar, less than or about 5 mbar, less than or about 2.5 mbar.

Because the formulation recirculates, and there is no concern formulation will leak out of the nozzle holes of aerosolizer 1201, the system becomes insensitive to the rate of pumping of pump 1207 and the duration of pumping of pump 1207, and the pumping becomes decoupled from the rate and duration of aerosolization by aerosolizer 1201. Because of this, pump 1207 can pump for as long as required (including before the start of aerosolization or after the aerosolization has concluded) at essentially any rate of flow that will eliminate most or all of the air from the formulation. When formulation is aerosolized, and when air exits through porous hydrophobic element 1303, the volume of formulation in the loop goes down, and thus the pressure in the loop goes down. The reduced pressure in tube 1305 draws formulation from formulation chamber 1213 via tube 1210. Because the air is introduced into the formulation during the aerosolization process and then essentially completely removed by debubbler 1304, the amount of formulation drawn out of formulation chamber 1213 is essentially equal to the amount of formulation aerosolized.

Small hole 1221 allows the pressure in aerosol head cavity 1222 and formulation cavity 1224 to equilibrate with the outside air, while being small enough maintain a high relative humidity in aerosol head cavity 1222 and formulation cavity 1224, thereby reducing evaporation of formulation carrier through hydrophobic porous membrane 1303. Because flexible formulation chamber 1213 is equilibrated with the outside air, the formulation in the device is maintained substantially at the same pressure as the outside air, substantially reducing or eliminating pressure differences across the nozzle holes of aerosolizer 1201, for example due to weather or changes in altitude, that can cause leakage of formulation or air ingress. In a related embodiment, debubbler 1304 removes air from the formulation into a small debubbler cavity, which debubbler cavity vents directly to the outside air, or to aerosol head cavity 1222, or directly to formulation cavity 1224, preferably through a small hole as described above to maintain high relative humidity in the debubbler cavity and thereby reduce evaporation of formulation carrier through porous hydrophobic element 1303. In the event that formulation does exit the debubbler, optional absorbent material 1223 may be used to contain the leaked formulation. Preferably absorbent material 1223 is not required.

FIG. 13 shows valve 1209 at the exit of formulation chamber 1313. Valve 1209 may be a controlling valve such as a needle valve or the like. Preferably, valve 1209 has only two states, open and closed. In one embodiment, during shipping and storage, formulation chamber 1213 contains all of the formulation in the cartridge, and the exit of formulation chamber 1213 is closed by valve 1209. Preferably valve 1209 closes the exit of formulation chamber 1213 by pinching off a section of formulation chamber 1213, which section is made of the same material(s) as the rest of formulation chamber 1213, which materials have been optimized for long term storage of the formulation. In this way, during transport and storage the formulation only contacts the surface of formulation chamber 1213, and not valve 1209 or any of the other flow components in the cartridge. In this way the materials of formulation chamber 1213 can be optimized for drug contact, and the materials of the other components can be optimized for performance. Before the first use of the current invention, valve 1209 is opened, and pump 1207 self-primes and then pumps the formulation though the recirculation system for sufficient time that essentially all of the air in formulation pocket, tubes 1210, 1211, 1315, and 1305, debubbler 1304, and pump 1207 is forced out of the cartridge, via aerosolizer 1201 and/or debubbler 1304. To facilitate priming of pump 1207, a similar valve may be placed on 1305 to ensure that while priming, pump 1207 does not simply pump air around the recirculation loop.

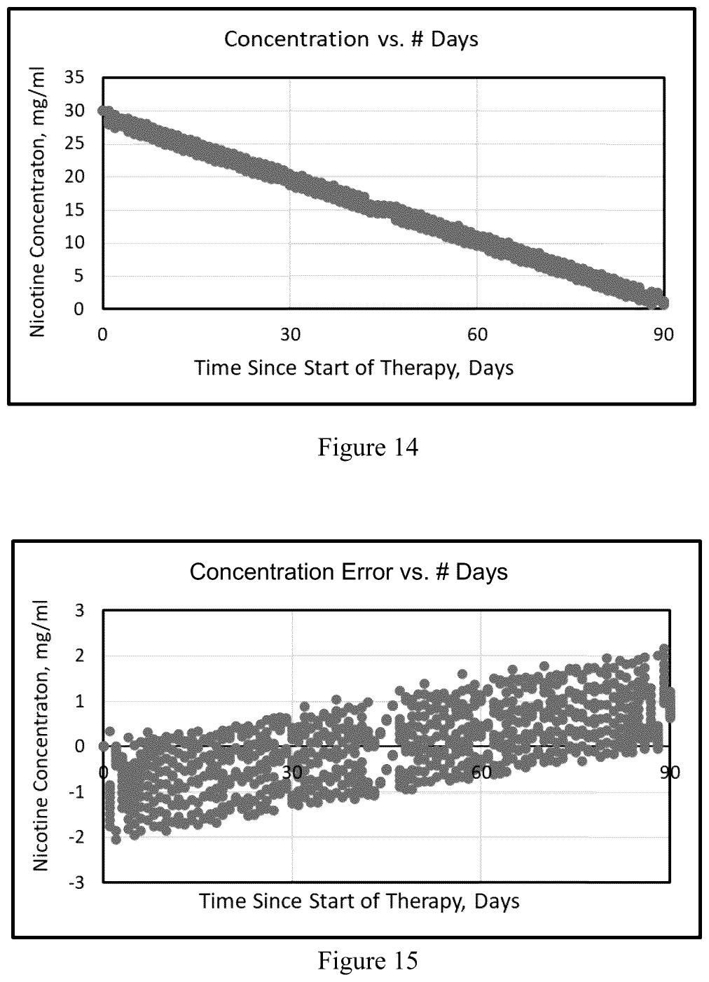

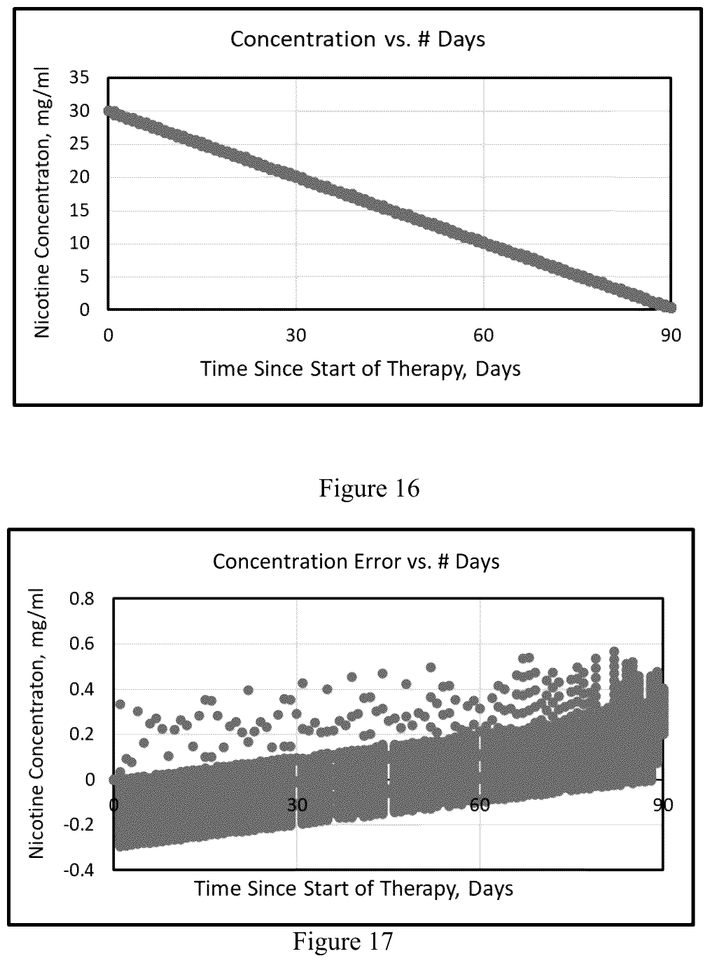

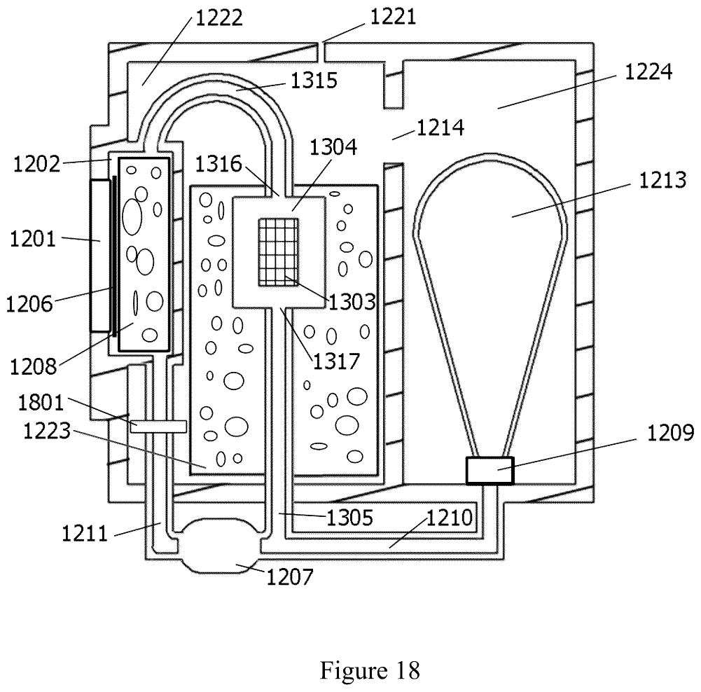

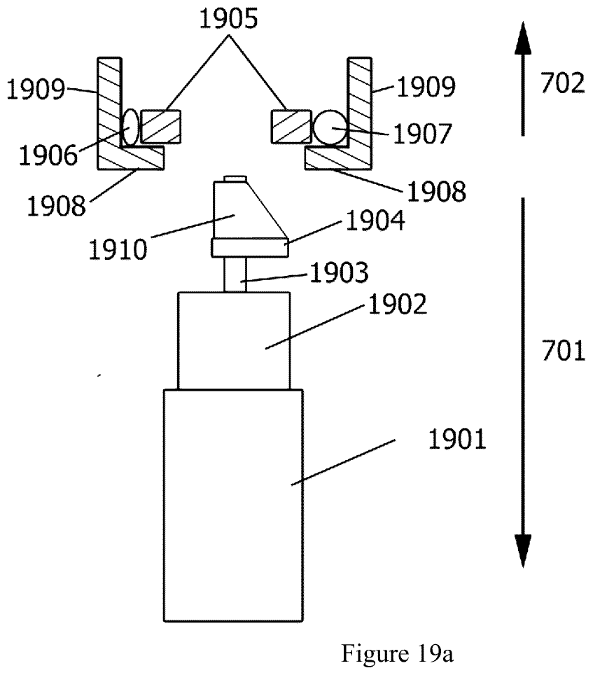

In the embodiment where there are two or more formulation chambers 1213, two or more valves 1209 can serve to control the ratios of the two or more formulations, for example reducing the concentration of the nicotine. This can be done by changing the amount of restriction to flow through the multiplicity of valves 1209. However, a preferred method is to only have one of the valves 1209 open at any time, for example during a dosing event. In this way the formulation mixture ratio can be controlled. FIGS. 14 through 17 show numerical simulations of this method as applied to two formulation chambers, one with sham formulation, and the other with nicotine at a concentration of 30 mg/ml. In FIGS. 14 through 17, it is assumed that 1) a user uses the device 10 times (10 “dosing events”) every day, 2) takes 7 puffs in each dosing event, and 3) the target of the algorithm is to ramp the concentration of nicotine down substantially linearly to substantially zero over a period of 90 days. The algorithm used is to calculate the current concentration based on previous dosing events. If the concentration is calculated to be above the target concentration, a dose (either a dosing event or a puff) is conducted, drawing formulation from a “sham” (zero concentration) formulation chamber 1213. If the calculated concentration is below the target concentration, and dose is conducted drawing formulation from a formulation chamber 1213 that contains an active pharmaceutical ingredient, preferably nicotine. FIG. 14 presents the calculated concentration of nicotine delivered as discussed above, where the formulation chamber from which formulation is drawn was selected at the beginning of each dosing event. FIG. 15 presents the error (difference from the target nicotine concentration) of each dose delivered. It can be seen that wish this algorithm, nicotine can be controlled to within ±2 mg/ml, or ±6.7% of the initial target concentration of nicotine. It can be seen that by taking out the linear trend with the algorithm, the concentration can be controlled to about ±1.5 mg/ml. FIGS. 16 and 17 are similar to FIGS. 14 and 15, except the selection of which formulation chamber 1213 to draw formulation from is done before each puff. It can be seen from FIG. 17 that when using this algorithm, the concentration can be held to about ±0.45 mg/ml, or ±1.5%.

Preferably aerosolizer 1301 creates an aerosol into the inhalation airflow of the user as the user inhales through the device. In prior art devices using a vibrating mesh nebulizer, the aerosol is generated in a direction toward the exit of the device and toward the user. In one embodiment, the airflow through the airway flows in a direction which is substantially perpendicular to the average direction of aerosol generation from the array of holes, and substantially parallel to aerosolizer 1301. It has been found that this configuration more efficiently entrains the dose and disperses the aerosol, reducing aggregation of aerosol particles, thereby increasing emitted dose and reducing particle size.

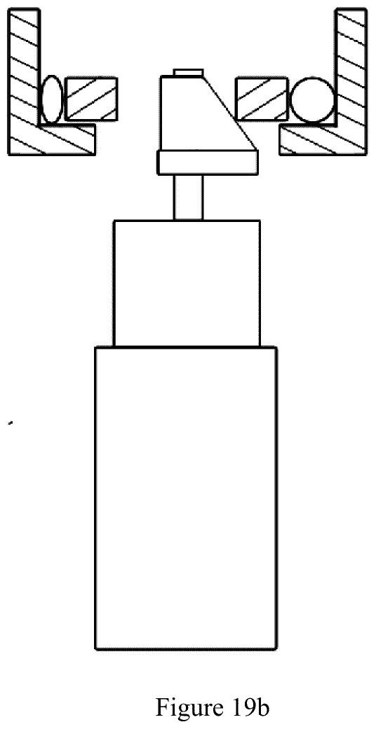

FIG. 18 shows an embodiment of recirculation system with filter 1801. In the embodiments of current invention where the aerosolizer is comprised of one or more nozzle holes, there is a concern that bacteria might enter through the nozzle holes and colonize the formulation in the recirculation loop. It is also possible for bacteria to enter through porous hydrophobic element 1303, although this is less likely because it is only exposed via small hole 1221. Viruses are much less of a concern as they can only reproduce in a host cell, not in water-based formulations. Any virus exposure to a nozzle hole is most likely from air being inhaled through the airway, leading to much larger exposure to a user from them simply breathing the air than they could get from any exposure to viruses that get into the formulation and then are inhaled. In one embodiment, the formulation has a bacterio-static agent to stop bacteria from reproducing. However, it is preferred to no have a bacteriostatic agent in the formation so as to not exposure the user's lungs to this agent. In the embodiment of FIG. 18, the system is supplied with filter 1801. Filter 1801 preferably has a pore size that will capture essentially all bacteria that might get into the formulation. Preferably the pore size of filter 1801 is less than or about 1 μm, less than or about 0.75 μm, less than or about 0.5 μm, less than or about 0.4 μm, less than or about 0.2 μm, less than or about 0.1 μm. Preferred filters 1801 have a pore size selected from about 0.1 μm, 0.2 μm, and 0.22 μm. In one embodiment, the filter 1801 used is a 0.2 μm pore size nylon membrane filter from Omicron part number 170047R. The filter may be placed anywhere in the recirculation loop, for example tube 1215, 1210, 1205, or 1211. However, it is preferred that filter 1801 not be placed between the exit of 1202 and before the entrance to pump 1207, as the relatively high pressures required to pump though filter 1801 due to the small pore size and limited area available for filter 1801 could cause formulation to leak out of the nozzle hole or holes of aerosolizer 1201 or out of the porous hydrophobic component 1303. Preferably the filter is placed between pump 1207 and the inlet to formulation pocket 1202, either in tube 1211 or preferably at the inlet to formulation pocket 1202. With filter 1801 in place, any time pump 1207 pumps formulation, formulation pocket 1202 is supplied with bacteria-free formulation, and any bacteria in pocket 1202 will be swept into tube 1215, and ultimately pumped around the recirculation loop to be captured by filter 1801. In one embodiment, the system has a mode wherein pump 1207 pumps a sufficient volume such that essentially all of the formulation initially in formulation pocket 1202 will flow out of pocket 1302 and be replaced by bacteria free formulation, resulting in an aseptic fill of pocket 1202. Preferably, the sufficient volume is greater than or about equal to the volume of pocket 1202, greater than or about 1.5 times the volume of pocket 1202, greater than or about 2 times volume of pocket 1202, greater than or about 2.5 times to the volume of pocket 1202, greater than or about 3 times to the volume of pocket 1202, greater than or about 4 times to the volume of pocket 1202, greater than or about 5 times to the volume of pocket 1202, greater than or about 7.5 times to the volume of pocket 1202, or greater than or about 10 times to the volume of pocket 1202. Preferably, the sufficient volume is pumped by pump 1207 at a time selected from one or more of the list including but limited to over the course of a day, before the first dose of the day, before each dosing event, after each dosing event, before each puff, after each puff. It is important to be sure that filter 1801 is intact, for example has no rips or leaks, and has the correct pore size. This can be determined by measuring the pressure required to achieve a predetermined flow rate, for example having a pressure transducer in tube 1211 between pump 1207 and filter 1801. Reduction in the pressure due to a failure of filter 1801 may lead to a faster pumping rate, in which case the failure may be detected with a flow meter. The flow meter should be placed between the outlet of pump 1207 and the inlet of filter 1801, as any other location in the flow loop may not be able to detect a small leak associated with filter 1801. In the embodiment where pump 1207 is driven by a motor, for example where pump 1207 is a peristaltic pump, a change in pumping rate could be detected as a change in motor speed. For a pump motor run at constant voltage, an increase in pump speed could be detected as a decrease in current due to the increase in the motor back emf. For a pump motor run at constant current, an increase in flow rate would result in an increase in required voltage to maintain the current. For a pump controlled with a pulse width modulated scheme using an encoder to run at constant pump motor speed, a decrease in pressure would result in a shorter required pulse width to maintain the same speed.

Pump 1207 may be any type of pump capable of generating a sufficient output pressure to achieve a sufficient flow rate, for example through filter 1801. Preferred pumps can generate high output pressures to enable pumping through 1801. A particularly preferred pump is a peristaltic pump comprised of components selected from but not limited to a motor with 148 RPM at 12V, a gear head, a rotor or eccentric, a ring, a section of flexible tubing, and a support surface for the tubing. Preferred motor and gearhead result in an output shaft rotation rate of from about 50 to about 1,000 RPM, from about 75 to about 750 RPM, from about 100 to about 500 RPM, from about 125 to about 250 RPM, from about 125 to about 200 RPM, or from about 125 to about 175 RPM. Preferably, the motor, gearhead, and rotor or eccentric ring are part of durable system 701, and the tubing, ring if required, and support surface are part of cartridge 702. In one embodiment of the peristaltic pump a motor turns a rotor, which rotor circularly translates at least one roller, preferably two or more or three or more rollers, whereupon the rollers roll along a piece of flexible tubing and compresses it against a supporting surface in order to create flow through the tubing. Because the tubing needs to be compressed in order for the rollers to be compressed, preferably each roller is supplied with an angled cam surface that compresses the tubing.

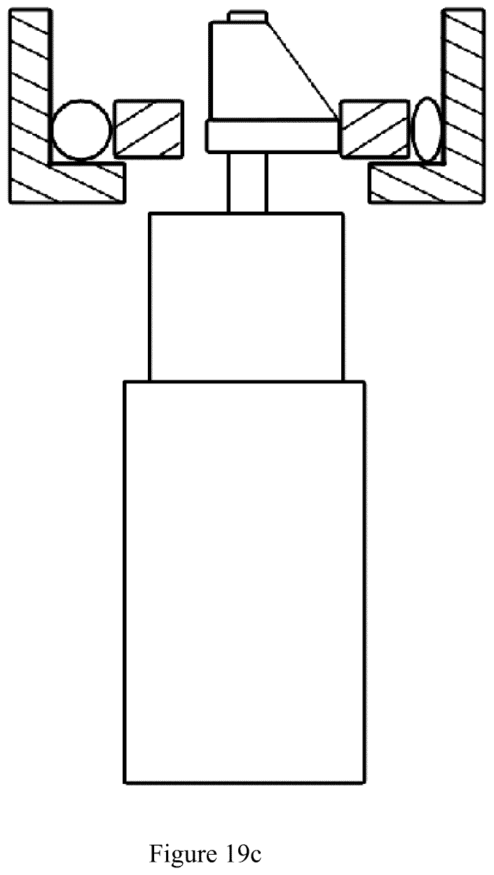

FIG. 19 shows a preferred embodiment of pump 1307, wherein the peristaltic pump is comprised of motor 1901, gear head 1902, shaft 1903; and eccentric cam 1904 and alignment cam 1910; both of which are mounted on shaft 1903, and all of which are parts of durable system 701. Ring 1905, tubing 1907, shown in FIG. 19a as compressed tubing 1906 and uncompressed tubing 1907, ring capture detail 1908, and support surface 1909 which are all part of cartridge 702. The cartridge components are shown in cross section, it will be understood that as viewed from the top, ring 1905 is circularly symmetric, as are support surface 1909 and tubing 1907 except where tubing 1906 is compressed by ring 1905 and where there is a gap in support surface 1909 where input and output sections of tubing 1907 feed in. Preferably at least one of eccentric cam 1904, alignment cam 1910, and ring 1905 are made of a polymer with properties selected from a list including but not limited to a lubricious polymer, a fluorinated polymer, comprised of PTFE, comprised of Delrin, or comprised of a nylon. In an alternate embodiment, one of eccentric cam 1904, alignment cam 1910, and ring 1905 are made of metal, preferably a stainless steel, and ring 1905 is a radial ball bearing.

FIG. 19a show the pump configuration when a new cartridge 702 is ready to be installed. It can be seen in 19a that eccentric cam 1904 is to the right, but ring 1905 is to the left. In general the relative positions of eccentric cam 1904 and ring 1905 are arbitrary, but it is unlikely that they will be aligned. Without alignment cam 1910, it can be seen that ring 1905 and eccentric cam 1904 will interfere when cartridge 702 is being installed, potentially leading to breakage of ring 1905 and/or eccentric cam 1904. To avoid this, alignment cam 1910 is installed on shaft 1903 above eccentric cam 1904. As shown in FIG. 19b, as cartridge 702 is being pressed onto durable system 701, alignment cam 1910 contacts ring 1905 and begins moving ring 1905 to the left. As can be seen in FIG. 19c, when cartridge 702 is fully installed, ring 1905 has been moved to the right, and now the tubing is compressed to the right. When motor 1901 is energized, eccentric cam 1904 is caused to rotate, and sections of tubing 1907 are sequentially compressed, causing flow of the formulation contained in tubing 1907.

When cartridge 702 is attached to durable system 701, the eccentric slides into the ring, characterized by being of sufficient diameter that the eccentric fits completely with in the ring, and whereupon activation of the motor the eccentric rotates the eccentric and thereby translates the ring, causing the ring to sequentially compress sections of the tubing against a supporting surface in a circular pattern, thereby creating flow through the tubing. In an embodiment wherein the system is comprised of a durable system element and a separable multidose cartridge, it is preferable that the motor, gear head, and eccentric or rotor are part of the durable system element and the tubing, ring, and support surface are part of the disposable.

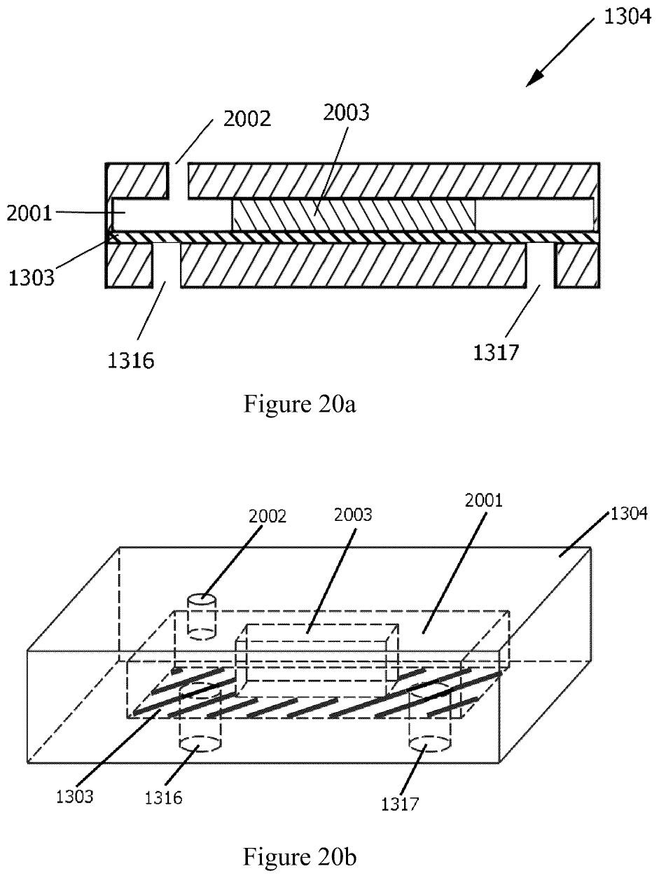

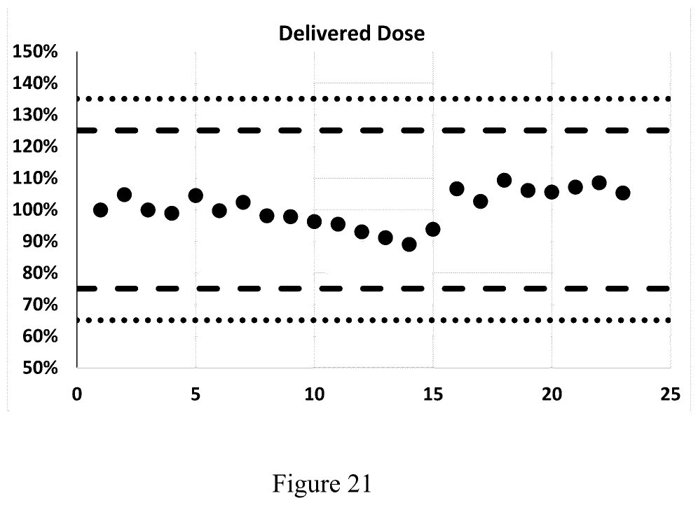

FIG. 20a shows an embodiment of debubbler 1304 in cross section, and FIG. 20b show a perspective drawing of the same embodiment. Formulation and air flow into debubbler 1304 via entrance channel 1316, whereupon the formulation comes into contact with porous hydrophobic material 1303, lifts and then travels under porous hydrophobic material 1303, and at least a portion of the air in the formulation exits through the pores in porous hydrophobic material 1303 and into debubbler cavity 2001. The formulation then exits debubbler 1304 through exit channel 1317. Optional divider or dividers 2003 separate debubbler cavity 2001 into two or more channels, reducing the unconstrained span of porous hydrophobic material 1303, resulting in less lifting of hydrophobic material 1303, thereby increasing the likelihood that an air bubble will come into contact with porous hydrophobic material 1303. Exit channel 1317 is somewhat restrictive, creating sufficient pressure under porous hydrophobic material 1304 to drive air through the pores of porous hydrophobic material 1303. Preferred gauge pressures for the formulation and air under porous hydrophobic material 1303 are from about 10 to about 1000 mBar, from about 20 to about 500 mBar, from about 30 to about 250 mBar, from about 40 to about 100 mBar, from about 50 to about 80 mBar, or about 10, 20, 40, 80, 160, 320, or 640 mBar. The air that exits through porous hydrophobic material 1303 into debubbler chamber 2001 exits through small hole 2002. Small hole 2002 is a small hole as described previously, and its diameter is chosen such that very little water vapor escapes and thus the relative humidity in debubbler chamber 2001 remains close to 100%, limiting the evaporation of carrier through the pores of porous hydrophobic material 1303. Preferably the evaporation through hydrophobic material 1303 is less than about 10 μl/hour, less than about 5 μl/hour, less than about 2.5 μl/hour, less than about 1 μl/hour, or less than about 0.5 μl/hour. A desired hydrophobic porous material used for 1303 is POREX MD10 expanded PTFE with typical airflow through the pores of 125 (min 70) l/hr/cm2 at 70 mbar and water entry pressure (WEP) of 270 (min 175) mbar. The airflow of POREX expanded PTFE can range from 2-125 l/hr/cm2 at 70 mbar and the WEP can range from 175-1050 mbar so various embodiments of this system can use expanded PTFE with airflows and WEP that output the desired airflow based on the internal pressure of the system. Additional hydrophobic porous materials that can be used are Versapor RC Membranes including but not limited to Versapor 200RC part number VRC02S7X10, Versapor 800RC part number VRC08S7X10, Versapor 1200RC part number VRC12S7X10, Versapor 3000RC part number VRC30S7X10, Versapor 5000RC part number VRC50S7X10.

### D. Pharmaceutical Agents Used in the Aerosol Device

The active pharmaceutical ingredients (API) encompassed by the present disclosure include nicotine as the free-base and pharmaceutically acceptable salts thereof. In one embodiment, the pharmaceutical agent used in the aerosol device is nicotine bitartrate dihydrate. Other acids that can be mixed with the free-base include, but are not limited to, tartaric, lactic, benzoic, levulinic, salicyclic, and malic acids. The source of nicotine of the nicotine-containing compositions of the invention can include nicotine in free base form, salt form, as a complex, as a solvate, or other suitable form. Nicotine is typically isolated (e.g., as described above) in neat (liquid) form. According to the present invention, nicotine is modified such that it is provided in other forms by incorporating the nicotine as a component of a salt, co-crystal, or salt co-crystal, e.g., in the form of an oil, solid, semi-solid, etc. In some embodiments, certain salts, co-crystals, and salt co-crystals are desirably provided in solid form, e.g., solid, crystalline form. Advantageously (although not necessarily), coformers (including acids) that are combined with nicotine to form such nicotine salts, co-crystals, or salt co-crystals are “GRAS” (Generally Regarded As Safe) according to the U.S. Food and Drug Administration. Furthermore, it is beneficial (although again, not necessary) for the nicotine salts, co-crystals, and/or salt co-crystals produced thereby to also be GRAS. When nicotinic compounds of the present invention contain relatively basic functionalities, as in nicotine, for example, acid addition salts can be obtained by contacting the neutral form of such compounds with a sufficient amount of the desired acid, either neat or in a suitable inert solvent. Exemplary pharmaceutically acceptable nicotine salts include tartrate (e.g., nicotine tartrate and nicotine bitartrate), chloride (e.g., nicotine hydrochloride and nicotine dihydrochloride), sulfate, perchlorate, ascorbate, fumarate, citrate, malate, lactate, aspartate, salicylate, tosylate, succinate, pyruvate, and the like; nicotine salt hydrates (e.g., nicotine zinc chloride monohydrate), and the like. One skilled in the art will appreciate that analogous salts can be formed for agonist compounds comprising relatively basic functionalities. Additional acids that can form salts include formic, acetic, propionic, isobutyric, butyric, alpha-methylbutyric, isovaleric, levulinic, beta-methylvaleric, caproic, 2-furoic, benzoic, phenylacetic, heptanoic, octanoic, nonanoic, oxalic, malonic, glycolic acid, benzenesulfonic, camphosulfonic, ethanesulfonic, gluconic, glucoronic, glutamic, hippuric, hydrobromic, isethionic, lactobionic, maleic, mandelic, methanesulfonic, mucic, naphthalenesulfonic, nicotinic, nitric, pamoic, pantothenic, phosphoric, sulfuric and the like as well as other fatty acids having carbon chains of up to about 20 carbon atoms. The inhalable substance medium can further comprise, for example, one or more of glycerin, water, and a flavorant. The amount of nicotine salt or polymorph form incorporated can vary and, in some embodiments, can be that amount sufficient to provide nicotine in an amount of about 0.01 mg to about 0.5 mg, about 0.05 mg to about 0.3 mg, or about 0.1 mg to about 0.2 mg per puff on the device.

Non-nicotine containing formulations for smoking cessation are also covered under this disclosure. One such example is a formulation of the following comprising ascorbic acid (3 mg), mentha crispa (1.5 mg), Mentha piperita (1 mg), chocamine (1 mg), damiana leaf 10:1 P.E. (1 mg), palmitoylethanolamide (600 mcg), nicotinic acid (500 mcg), 5-hydroxytryptophan (500 mcg), nicotine adenine mononucleotide (100 mcg), zeaxanthin (100 mcg), lutein (50 mcg), lycopene 5% (50 mcg), water (1 mL), lecithin (0.04 mL) wherein the formulation has a total volume of greater than or about 0.1 ml, 0.5 ml, 1 ml, 2.5 ml, 5 ml, 10 ml, 15 ml, or 20 ml.

Other active pharmaceutical ingredients contemplated by the present disclosure include an opioid (e.g, fentanyl, morphine, hydrocodone, oxycodone, oxymorphone, codeine, hydromorphone, tapentadol), a triptan, risperidone, insulin, epinephrine, atropine, ciprofloxacin, methocarbamol, a benzodiazepine, a PDE5 inhibitor, a glucagon-like peptide, an API for cramps, and API for insomnia, a vasodilator, an API for asthma, an API for COPD, an API for depression, an API for PTSD, an API for depression, an API for angina, an API for hypoglycemia, an API for osteoporosis, an API for migraine, an API for nausea, an API for anaphylaxis, an API for poisoning, and an API for sexual dysfunction.

### E. Formulations

The formulations used in the device herein and delivered to patients can take a variety of different forms. Generally, such forms are sterile solutions or suspensions that are non-pyrogenic and formulated to be suitable for oral inhalation. The inhaled particles should be small in size (e.g., 0.5 to 10, preferably 0.5 to 5 micrometers).

Concentrations of active ingredients used in the present disclosure may be selected to be close to the solubility limit so as to reduce the volume of formulation per puff that needs to be aerosolized, increasing the lifetime of the cartridge, and the time between battery charges. However, the concentration should not be so high that slight changes in concentration, temperature, etc. cause the active pharmaceutical ingredient to come out of solution. For example, for nicotine bitartrate dihydrate, the solubility limit is about 50 mg/ml. Preferred concentrations of nicotine free base for use in the device of the present disclosure include, but are not limited to, about 5 mg/ml or more, about 10 mg/ml or more, about 15 mg/ml or more, about 20 mg/ml or more, about 25 mg/ml or more, about 30 mg/ml or more, about 35 mg/ml or more, about 40 mg/ml or more, or about 45 mg/ml or more. Depending on the salt of nicotine employed, the amount of such salt can be adjusted to provide the desired dose of nicotine free base, i.e., the amount of free base can be calculated from the mass of nicotine salt present in the formulation.

In one embodiment, the formulation comprises nicotine free base in an amount of about 20 mg/ml to 50 mg/ml, or about 25 mg/ml to 35 mg/ml and 0.9% sodium chloride solution, and optionally other excipients. In another embodiment, the formulation consists essentially of nicotine free base in an amount of about 20 mg/ml to 50 mg/ml, or about 25 mg/ml to 35 mg/ml and 0.9% sodium chloride solution. The volume of the nicotine-containing solution delivered per actuation of the device of the present disclosure may be about 1 to 50 microliters, or about 1 to 30 microliters, or about 1 to 25 microliters, or about 1 to 20 microliters, or about 1 to 15 microliters, or about 5 to 20 microliters, or about 10 to 20 microliters. Such volume is delivered by the device of the present disclosure in about 0.1 to 2.5 seconds, or about 0.25 to 2 seconds, or about 0.5 to 1.5 seconds, or about 0.75 to 1.25 seconds, or about 1 second.

For highly potent active ingredients, for example fentanyl, it may be desirable to reduce the concentration for safety so that there is no need to aerosolize very small volumes. For example, the concentration of fentanyl may be less than or about 10 mg/ml, 5 mg/ml, 2.5 mg/ml, 1 mg/ml, 0.5 mg/ml, 0.25 mg/ml, 0.1 mg/ml, 0.05 mg/ml, or 0.025 mg/ml. In one embodiment, the concentration is from about 0.5 to about 1 mg/ml, or 0.05 to about 0.5 mg/ml, or about 0.1 to about 0.3 mg/ml.

The formulations may contain pharmaceutical excipients that can either improve the stability of the dosage form or provide comfort during administration. Such excipients include carriers (e.g., water) and pharmaceutical solvents, tonicity adjusting agents, pH adjusting or buffering agents, stabilization agents, antimicrobial preservatives, dispersing and/or wetting agents.

Pharmaceutical solvents are used to dissolve or disperse pharmaceutically active medicaments and excipients. The solvent may be aqueous or non-aqueous. A formulation of the present invention may be formulated with one or a mixture of more than one pharmaceutically acceptable solvent and is selected from, but not limited to, glycerol, propylene glycol, polyethylene glycol, polypropylene glycol, ethyl alcohol, isopropyl alcohol, water, mineral oil, peanut oil, and corn oil. The pharmaceutical solvents may be used to prepare the formulation concentrate as well as used for reconstitution of the dosage form of the present invention. Pharmaceutically acceptable solvents such as water, ethyl alcohol, isopropyl alcohol are evaporable and are usually used to dissolve or disperse the medicament and excipients in the formulation concentrate. Glycerol, propylene glycol and polyethylene glycol are co-solvents and are used to assist in solubilization of water insoluble or poorly water soluble medicaments in the formulation concentrate. Pharmaceutically acceptable reconstituting solvents such as sterile water for injection, sterile normal saline solution, sterile phosphate buffer solution and sterile 5% dextrose solution are used for reconstitution of the dosage form of the present invention to form a solution or a fine particle suspension of pharmaceutically active ingredient prior to oral inhalation via a nebulizer or aerosol device. The reconstituting solvents may be packaged in individual ampoules or unit dose plastic containers for unit of use or may be packaged in large volume sterile container from which a specific volume of the reconstituting solvent can be withdrawn without contaminating the solvent.

Tonicity-adjusting agents are used to enhance the overall comfort to the patient upon administration of the reconstituted dosage form. It is preferred to adjust the osmolality of the reconstituted inhalation solution to about 275 to 305 (range 254 to 325) mOsm/Kg. Tonicity-adjusting agents for inhalation use are sodium chloride, dextrose, lactose, sodium phosphate, sorbitol, mannitol and sucrose or combination thereof at a concentration to generate an isotonic solution after the dosage form is reconstituted with 1 to 10 ml of sterile water for nebulization. The addition of sugars such as dextrose, lactose and sucrose adds stickiness and adherent characteristics to the formulation so that the dried-medicament and formulation excipients can be better retained on the supporting material after the formulation concentrate is dried. The tonicity-adjusting agent can also function as a particle partition agent to reduce particle size of the pharmaceutically active ingredient after the formulation is impregnated in or deposited on the supporting material and to assist in dissolution or dispersion of pharmaceutically active ingredient particles upon reconstitution with the pharmaceutical solvent. Alternately, the dosage form may be formulated without the addition of a major tonicity-adjusting agent. The desired tonicity of the dosage form is achieved by reconstituting with a sterile isotonic saline solution.

The formulations may also comprise pH adjusting or buffering agents to adjust or maintain the pH of pharmaceutical dosage form to a desired range for the following reasons: (1) To provide an environment for a better product stability that pharmaceutical active ingredient may express a better chemical stability within certain pH range; (2) to provide better comfort for the patient at administration; and (3) to provide a pH range for better antimicrobial preservative activity. Formulations and dosage forms of the present invention may be formulated with one or more pharmaceutically acceptable pH adjusting or buffering agents so that, after reconstitution, the desired pH is between about 3 to about 8.

Pharmaceutically acceptable pH-adjusting and buffering agents are selected from, but not limited to, hydrochloric acid, sulfuric acid, nitric acid, acetic acid, phosphoric acid, fumaric acid, citric acid, tartaric acid, maleic acid, succinic acid, ammonia solution, ammonium carbonate, sodium borate, sodium carbonate, triethanolamine, trolamine and sodium hydroxide. Stabilizing agents are antioxidant and chelating agents that are capable of inhibiting oxidation reaction and chelating metals, respectively, to improve stability of pharmaceutically active ingredient and excipients.

Formulations and dosage forms of the present invention may be formulated with one or more pharmaceutically acceptable stabilization agents at a concentration suitable for the intended pharmaceutical applications, and may be selected from, but not limited to, chelating agents such as EDTA and its sodium salt, citric acid and sodium citrate, anti-oxidation agents such as Vitamin E, ascorbic acid, ascorbyl palmitate, butylated hydroxyanisole, butylated hydroxytoluene, hypophosphorous acid, monothioglycerol, propyl gallate, sodium bisulfite, sodium metabisulfite, sodium formaldehyde sulfoxylate, and thiourea. The addition of a stabilizing agent to a dosage form of the present invention can improve stability of the pharmaceutical active substance and prolong the shelf life.

Antimicrobial preservative agents are used in pharmaceutical preparations to inhibit the growth of microorganisms. Dosage forms of the present invention may be formulated with one or more pharmaceutically acceptable anti-microbial preservatives at suitable concentrations to prevent microbial growth. Examples of pharmaceutically acceptable preservatives suitable for oral or nasal inhalation include, but are not limited to, parabens, benzalkonium chloride, benzethonium chloride, benzoic acid, sorbic acid or potassium sorbate, benzyl alcohol, cetylpyridinium chloride, chlorobutanol, phenol, phenylethyl alcohol, phenylmercuric nitrate, and thimerosal.

Wetting or dispensing agents are used to increase wettability and assist in dispersing of water insoluble or poorly water soluble particles. Examples of pharmaceutically acceptable wetting and dispersing suitable for oral or nasal inhalation agents are, poloxamers, oleic acid and its salts, lecithin and hydrogenated lecithin, sorbitan fatty acid esters oleyl alcohol, phospholipids including but not limited to phosphatidylglycerol, phosphatidylcholine and others, polyoxyethylene fatty alcohol ethers, polyoxypropylene fatty alcohol ether, polyoxyethylene fatty acid ester, glycerol fatty acid esters, glycolipid such as sphingolipid and sphingomyelin, polyoxyethylene glycol fatty acid ester, polyol fatty acid esters, polyethylene glycol glycerol fatty acid esters, polypropylene glycol fatty acid esters, ethoxylated lanolin derivatives, polyoxyethylene fatty alcohol, polyoxyethylene sorbitan fatty acid esters, polyoxyethylene stearate, propylene glycol alginate, dilauryldimethylammonium chloride, D-α-tocopheryl-PEG 1000 succinate, Polyoxy 40 stearate, polyoxyethylene-polyoxypropylene block copolymers, polyoxyethylene vegetable oils, fatty acid derivatives of amino acids, glyceride derivatives of amino acids, benzalkonium chloride, bile acids.

### F. List of Additional Numbered Embodiments

The additional numbered embodiments below will be understood to apply to any of the disclosure and claims in this application, and, in determining the subject matter encompassed by these embodiments, the references to the “system or device of any of the above embodiments” will be understood to also be a reference to any of the embodiments and claims below. Further, the following list of embodiments is intended to supplement the preceding description as well as the Examples and Claims that follow the list.

1. A smoking cessation system, comprising:
a. A durable system, comprising electronics and a peristaltic pump motor assembly;
b. A cartridge comprising an aerosolizer, a debubbler, a filter, a pump head and one or more formulation chambers; and
c. A wall charger and cable.

2. The system of No. 1, further comprising an app for a portable device that communicates with the system.

3. The system of any one of Nos. 1-2, wherein the cartridge is removably connected to the durable system.

4. The system of any one of Nos. 1-3, further comprising a controller and a mouthpiece.

5. A method of treating a patient in need of smoking cessation therapy, comprising:
a. Providing a system of any one of Nos. 1-4 wherein the cartridge comprises a nicotine formulation in the one or more formulation chambers;
b. Administering the nicotine formulation by the patient sealing her/his lips around the mouthpiece and inhaling a dose of the nicotine formulation, wherein the patient administers to herself/himself multiple gradually decreasing doses of the nicotine formulation over time.

6. The method of No. 5 wherein the period of time is about 2-6 weeks, or about 3-5 weeks, or about 4-5 weeks or about 4 weeks.

7. The method of Nos. 5-6, wherein the dose gradually decreases by about 1%-10% per day during the predetermined period of time.

8. The method of Nos. 5-7, wherein the nicotine formulation comprises nicotine free base of a pharmaceutically acceptable salt thereof.

9. The method of Nos. 5-8, wherein the nicotine formulation comprises about 5 mg/ml to about 50 mg/ml of nicotine free base.

10. The method of Nos. 5-9, wherein the nicotine formulation comprises about 20 mg/ml to about 50 mg/ml of nicotine free base and 0.9% sodium chloride solution.

11. The method of Nos. 5-10, wherein the system has means for delivering a volume of the nicotine formulation per actuation of about 1 to 50 microliters, or about 1 to 30 microliters, or about 1 to 25 microliters, or about 1 to 20 microliters, or about 1 to 15 microliters, or about 5 to 20 microliters, or about 10 to 20 microliters.

12. The method of Nos. 5-11, wherein the system has means for delivering a volume of the nicotine formulation per actuation of about 1 to 50 microliters, or about 1 to 30 microliters, or about 1 to 25 microliters, or about 1 to 20 microliters, or about 1 to 15 microliters, or about 5 to 20 microliters, or about 10 to 20 microliters, and wherein such volume is delivered by the system in about 0.1 to 2.5 seconds, or about 0.25 to 2 seconds, or about 0.5 to 1.5 seconds, or about 0.75 to 1.25 seconds, or about 1 second.

13. A smoking cessation inhaler, comprising: a durable part (or system or element) of the inhaler comprising a controller and a multidose cartridge configured to be separably attached to the durable part of the inhaler wherein the cartridge comprises (a) an aerosolizer; (b) a formulation chamber containing a formulation comprising an active pharmaceutical ingredient; (c) a debubbler; and (d) a filter.

14. The inhaler of No. 13, further comprising a pump motor assembly.

15. The inhaler of Nos. 13-14, further comprising a debubbler; a debubbler chamber; a pocket; an aerosolizer; a filter; and a pump head.

16. The inhaler of Nos. 13-15, wherein the formulation comprises about 5 mg/ml to about 50 mg/ml of nicotine free base.

17. The inhaler of Nos. 13-16, wherein the nicotine formulation comprises about 20 mg/ml to about 50 mg/ml of nicotine free base and 0.9% sodium chloride solution.

18. The inhaler of Nos. 13-17, further comprising means for delivering a volume of the nicotine formulation per actuation of about 1 to 50 microliters, or about 1 to 30 microliters, or about 1 to 25 microliters, or about 1 to 20 microliters, or about 1 to 15 microliters, or about 5 to 20 microliters, or about 10 to 20 microliters.

19. The inhaler of Nos. 13-17, further comprising means for delivering a volume of the nicotine formulation per actuation of about 1 to 50 microliters, or about 1 to 30 microliters, or about 1 to 25 microliters, or about 1 to 20 microliters, or about 1 to 15 microliters, or about 5 to 20 microliters, or about 10 to 20 microliters, and wherein such volume is delivered by the inhaler in about 0.1 to 2.5 seconds, or about 0.25 to 2 seconds, or about 0.5 to 1.5 seconds, or about 0.75 to 1.25 seconds, or about 1 second.

20. The inhaler of Nos. 13-19, wherein the inhaler delivers nicotine free base resulting in pharmacokinetic values that are within about 5%, or 10%, or 15%, or 20%, or 25%, or 30% of those reported in cigarette smokers, wherein the values are one or more of (a) concentration at 5 minutes, 10 minutes and 15 minutes; (b) Cmax; (c) Tmax; and (d) AUC.

21. The inhaler of Nos. 13-20, wherein a statistically significant number of patients who use the inhaler report a decrease of nicotine cravings as measured by the visual analog scale (VAS)-craving assessment.

22. The inhaler of No. 21, wherein the inhaler decreases cravings by about 30%, or 40%, or 50%, or 60%, or 70% compared to baseline.

23. The inhaler of Nos. 13-22, wherein the inhaler comprises only a single formulation and a single pump.

### G. Examples

The following examples are included to demonstrate certain embodiments of the present disclosure. Those of ordinary skill in the art should, however, in light of the present disclosure, appreciate that modifications can be made to specific embodiments that are disclosed and still obtain a like or similar result without departing from the spirit and scope of the invention. Therefore, all matter set forth is to be interpreted as illustrative and not in a limiting sense.

### Example 1

An embodiment of the use of the system for smoking cessation is as follows. The system is supplied in a box comprising a durable system section comprised of electronics and a peristaltic pump motor assembly in an overwrap; one or more individually wrapped cartridges comprised of an aerosolizer, a debubbler, a filter, a pump head, and one or more formulation chambers; a wall charger and cable, and instructions for use. The system also includes an app which is downloaded onto a portable device such as a smart phone or tablet. An embodiment of the use of the system of the current invention as follows. The user installs the app on their phone, and receives instructions on how to use the system (or optionally follows the paper instructions for use). Following the instructions, the user removes from the box and unwraps the durable system cartridge and discards the overwrap. A supplied charger is attached via a USB-C charging connector and plugged in until the battery is fully charged, as indicated by a light switching from red to green. A first disposable cartridge is removed from its overwrap and pressed onto the durable system section, until a de-tenting feature is felt and heard to click. When the cartridge is installed, the user presses a button on the app (or optionally the button on the device), and a controller begins a purging procedure entailing turning on the pump for a sufficiently long time to remove air from flow channels, a formulation pocket, and a debubbler. The controller monitors the motor current and determines that the cartridge has been installed properly and is pumping formulation. When priming is complete, the user presses the button on the device. After about a second, a green flashing light on the device illuminates, indicating that the device is ready to use.

The user then seals their lips around the mouthpiece and begins inhaling. The microcontroller reads the inhalation flow rate from a sensor, and while the flow rate is in the correct range, the controller causes a piezo to oscillate, generating an aerosol through a nozzle array into the user's inhalation flow. The green light is illuminated steadily when the inhalation is in the desired flow rate range, guiding the inhalation of the user. The aerosol is generated for a maximum of 0.5 second, less if the user stops inhaling. The pump is turned on when the aerosol first starts and stays on for a second after the end of aerosol generation, pushing air generated by the aerosolizer into the debubbler, and replenishing formulation that has been aerosolized. The above steps are repeated upon each time the user inhales through the device based on the desire of the user, for as long as the flashing green light is illuminated. If an inhalation is not sensed for 5 minutes, the flashing green light is extinguished, and the system is put into a sleep mode. The user then pushes the button and repeats the subsequent steps as desired. When the formulation in a formulation chamber is depleted after approximately 4 weeks (based on the frequency and amount of usage), the user receives a text message and pushing the button presents a flashing light that alternates green and red on the device, indicating that cartridge needs to be replaced. The user then removes the cartridge by pulling it away from the durable system section and disposes of it, removes the overwrap from the next cartridge, and attaches the cartridge as before, optionally while viewing the instructions. During this whole process, the controller adjusts the concentration of the active ingredient, slowly tapering the concentration down to essentially zero over the course of 3 months. During the course of therapy, when the battery becomes depleted, the user receives a text message and the light presents a flashing red light. The user attaches a charger to device and charges until the device displays a steady green light, indicating that charging is complete.

### Example 2

The embodiment shown in FIG. 12 was tested for emitted dose efficiency 23 times using the method of USP 601: Delivered Dose Uniformity. A formulation of 10× normal saline was used. The filter was washed with a predetermined amount of deionized water and emitted dose was quantified using an electrical conductance method. FIG. 20 shows the results. The emitted dose averaged 82.01% of the amount of formulation aerosolized. The acceptance criteria of USP 601 of ±25% and ±35% are included for reference.

### Example 3

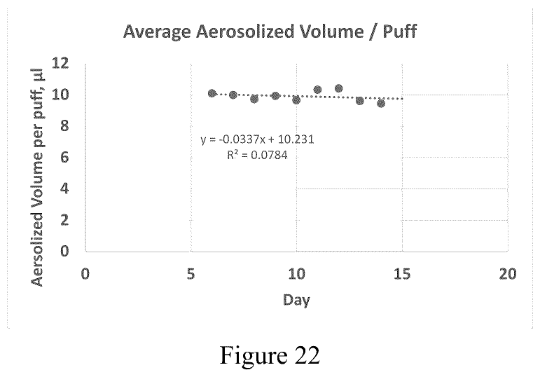

The embodiment of FIG. 13 was tested for aerosolized volume over a period of two weeks. Ten (10) dosing events separated by 90 minutes were performed each day, and each dosing event was comprised of 7 puffs separated by 30 seconds. A formulation of 3× normal saline was used. During the experiment, the system was not disturbed except to occasionally fill a graduated cylinder with formulation. The change of the volume of formulation in the graduated cylinder was used to determine the total aerosolized volume each day. The aerosolizer was configured before the start of the experiment to aerosolize 10 μl/puff. FIG. 22 presents the average aerosolized volume per puff for each of days 6 through 14. It can be seen from FIG. 22 that the embodiment of FIG. 13 is capable of delivering a repeatable aerosolized volume for at least a period of 2 weeks.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-101. (canceled)

102. An inhaler, comprising:
(i) a recirculation system comprising a first fluid passageway that extends from a formulation chamber to a pumping element;
(ii) a second fluid passageway that extends from the pumping element to a pocket directly behind an aerosolizer; and
(iii) a third fluid passageway that extends from the pocket back to the first fluid passageway.

103. The inhaler of claim 102, further comprising a filter placed between the pump and the pocket.

104. The inhaler of claim 103, wherein the filter has a pore size of about 0.1 μm to about 0.5 μm.

105. The inhaler of claim 104, wherein the pore size of the filter is chosen from about 0.1 μm, about 0.2 μm, and about 0.22 μm.

106. The inhaler of claim 105, further comprising a pump which pump is driven by a motor, wherein whether the filter is intact can be determined by one or more of: an increase in motor speed, a decrease in current, an increase in voltage, and a shorter required pulse.

107. The inhaler of claim 102, further comprising a debubbler.

108. The inhaler of claim 107, wherein the debubbler is a hydrophobic porous element.

109. The inhaler of claim 102, wherein the inhaler is comprised of a durable system device, and a multidose cartridge configured to be separably attached to the durable.

110. The inhaler of claim 109, further comprising a pump, wherein the cartridge contains components of a pump head, and the durable system contains a pump motor and a gear head.

111. The inhaler of claim 110, wherein the pump is a peristaltic pump.

112. The inhaler of claim 111, wherein the formulation chamber is flexible.

113. The inhaler of claim 112, further comprising a valve placed close to the outlet of the formulation chamber, wherein the valve is opened upon connection of the cartridge to the durable system device.

114. An inhaler, comprising:
(i) a nozzle array which is formed in a substrate; and
(ii) a sealing system with a compliant material on one end;
wherein the material is in contact with the substrate and blocks all or substantially all of the nozzles of the array when in the first position; and
further wherein the sealing system can be moved to a second position when the user is inhaling through the airway.

115. The inhaler of claim 114, further wherein the inhaler vibrates the substrate which contains the array of nozzle holes.

116. The inhaler of claim 115, wherein the inhaler creates an aerosol into the inhalation airflow of the user, and further wherein the airflow flows in a direction which is substantially perpendicular to the average direction of aerosol generation from the array of holes.

117. The inhaler of claim 116, further comprising a pressure sensor that detects a pressure drop in the airway when the user is inhaling, whereupon when the controller determines that a predetermined pressure drop has been exceeded, it actuates the aerosolizer and thus the generation of aerosol.

118. The inhaler of claim 117, wherein the controller senses the lack of sufficient inhalation airflow rate and stops the aerosolization.

119. The inhaler of claim 118, wherein the controller will restart aerosol generation if the user increases their inhalation rate and again exceeds the pressure threshold.

120. An inhaler, comprising:
A durable system device, and a multidose cartridge configured to be separably attached to the durable;
a formulation chamber containing a formulation comprising an active pharmaceutical ingredient, wherein the formulation chamber is flexible and collapses as formulation is drawn out of it; and
a valve placed close to the outlet of the formulation chamber, wherein the valve is opened upon connection of the cartridge to the durable system.

121. The inhaler of claim 120, wherein one or more of a filter, a debubbler, flexible tubing, a rotor, an eccentric element or cam, a ring, and a support surface, are components of the cartridge; wherein the drug contact surfaces are contained wholly in the cartridge.

122. The inhaler of claim 121, wherein a computing system controls an element chosen from a flow rate of one or more pumps, a state of one or more valves, a voltage supplied to the aerosolizer, the amplitude of oscillation of a component of the aerosolizer, a duration of pumping of one or more pumps, a duration of aerosolization of the aerosolizer, such that the amount of nicotine, or other active ingredient, delivered to the user is consistently lowered over time.

123. The inhaler of claim 122, wherein the element is a duration of aerosolization of an aerosolizer.
